(19) United States
(12) Patent Application Publication
(10) Pub. No.: US 2003/0059924 A1
(43) Pub. Date: Mar. 27, 2003

(54) Cloning of a novel inhibitor of antigen-receptor signaling by a retroviral-based functional screen

(76) Inventors: Helen Mancebo, Fremont, CA (US); Sacha J. Holland, San Francisco, CA (US); Jun Wu, Shanghai (CN); Charlene X. Liao, Palo Alto, CA (US)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/043,649

(22) Filed: Jan. 10, 2002

Related U.S. Application Data
(60) Provisional application No. 60/260,953, filed on Jan. 10, 2001.

(51) Int. Cl. C12N 1/20

(52) U.S. Cl. 435/252.3

(57) ABSTRACT

The present invention provides compositions and methods for modulating leukocyte and/or platelet activation. Nucleic acids and proteins which are capable of modulating leukocyte and/or platelet activation are provided. Compositions and methods for the treatment of disorders related to leukocyte and/or platelet activation are also provided. Prophylactics and methods for the prevention of such disorders are also provided. Also provided are compositions and methods for diagnostic and prognostic determination of such disorders. Further provided are assays for the identification of bioactive agents capable of modulating leukocyte and/or platelet activation.

This application is a continuation in part of U.S. provisional application No. 60/260,953, filed Jan. 10, 2001, which is incorporated herein in its entirety by reference.

### FIELD OF THE INVENTION

The present invention provides compositions and methods for modulating leukocyte and/or platelet activation. Nucleic acids and proteins which are capable of modulating leukocyte and/or platelet activation are provided. Compositions and methods for the treatment of disorders related to leukocyte and/or platelet activation are also provided. Prophylactics and methods for the prevention of such disorders are also provided. Also provided are compositions and methods for diagnostic and prognostic determination of such disorders. Further provided are assays for the identification of bioactive agents capable of modulating leukocyte and/or platelet activation.

### BACKGROUND OF THE INVENTION

Antigen receptors on T and B cells recognize pathogens and foreign antigens and initiate a series of intracellular biochemical events that result in complex biological responses. These signal transduction events are crucial for T and B cell development as well as T and B cell-mediated immune responses. For example, depending on the developmental stage of the cells, the dosage, type and duration of the antigen stimulus and the presence of co-stimulatory signals, engagement of antigen receptors can lead to either cell proliferation and differentiation or anergy and apoptosis. In the last decade, significant progress has been made towards understanding the mechanisms by which antigen receptors initiate such signaling cascades.

In the case of B lymphocytes, the B cell antigen receptor (BCR) complex consists of ligand-binding immunoglobulin (Ig) heavy and light chains, and the signal transducing Igα and Igβ co-receptors. The engagement of the BCR induces tyrosine phosphorylation of the immunoreceptor tyrosine based activation motifs (ITAM) present in the cytoplasmic tails of Igα and Igβ chains and activates multiple cytoplasmic protein tyrosine kinases (PTKs), including members of the Src, Syk and Btk families.

These early biochemical events, in turn, activate a number of downstream effector molecules, including PLCγ and guanine nucleotide exchange factors for Ras- and Rho-family GTPases. Activation of PLCγ leads to hydrolysis of PIP₂ to IP₃, which elevates the intracellular Ca⁺⁺ level, and diacylglycerol, which activates protein kinase C. Activation of Ras- and Rho-family GTPases regulates cytoskeletal rearrangement, and triggers mitogen-activated protein kinase (MAPK) pathways. The signaling cascades mediated by these secondary effectors ultimately lead to the modulation of gene transcription in the nucleus and determine the biological outcome of cellular activation.

It has become clear that the integration of distinct signaling cascades required for lymphocyte activation depends on the involvement of specific adapter proteins. Adapters are scaffolding proteins lacking enzymatic domains. However, they contain protein-protein interaction modules such as Src homology (SH) 2 and 3 domains, pleckstrin homology (PH) and WW domains. These modules specifically recognize and bind to discrete protein structures (SH2 domains recognize and bind to phospho-tyrosine in the context of a specific flanking sequence; PH domains interact with phospholipids; SH3 domains and WW domains associate with proline-rich regions), thus allowing formation of specific signaling complexes in the cell. For example, BLNK, a B cell-specific adapter contains a C-terminal SH2 domain and many tyrosine phosphorylation sites. Following BCR stimulation, the SH2 domain of BLNK interacts with the PTK Syk, which subsequently phosphorylates BLNK. The phosphorylation of BLNK on multiple tyrosine residues leads to recruitment of many critical SH2 domain-containing signaling proteins, including PLCg, Btk, Vav, and Nck. The importance of BLNK as a key scaffold for connecting different signaling cascades is further underscored by the complete blockage of B cell development and BCR-mediated effector function in both BLNK-deficient mouse and cell lines.

Although significant progress has been made in the past few years in dissecting the early biochemical events initiated by antigen receptors, the exact molecular mechanisms by which these early signaling events ultimately lead to downstream gene activation remain poorly understood. Since an improved understanding of such signaling pathways may help to uncover better therapeutic targets for inhibiting lymphocyte activation, as desired for fighting organ rejection and autoimmune diseases, as well as better therapeutic targets for promoting or enhancing lymphocyte activation, as desired in immunodeficiency disorders such as AIDS, it is of great interest to identify novel key proteins that regulate these complex processes. As an alternative to the traditional coimmunoprecipitation and protein purification approach, we established a large-scale functional genetic screening method using a retroviral-delivery system, which allowed efficient introduction of cDNAs into T and B cells and regulatable expression of the transgenes. Following multiple rounds of enrichment of the desired phenotype by fluorescence based sorting, we isolated a cDNA encoding a novel adapter molecule, named SLIM (for Src-like inhibitory molecule). SLIM (alternatively and equivalently referred to herein as SLAP-2) is related to Src-like adaptor protein (SLAP) (Pandey et. al., J. Biol. Chem., 270:19201-19204, 1995), which comprises N-terminal SH2 and SH3 domains and has previously been described as a negative regulator of T cell receptor (TCR) signaling, though SLAP is also expressed in B cells (Sosinowski et. al., J. Exp. Med., 191:463-474, 2000).

As demonstrated herein, overexpression of SLIM markedly impairs antigen receptor-mediated signal transduction leading to the blockage of antigen induced surface marker expression and induction of NFAT activity. SLIM may play an important role in negatively regulating T and B cell-mediated effector function and is a useful therapeutic target for the modulation of lymphocyte activation. Moreover, the retroviral-based functional screening methods described herein are shown to be efficient alternatives to traditional biochemical approaches for discovering key regulators in many signal transduction pathways.

### SUMMARY OF THE INVENTION

The present invention provides compositions and methods for modulating leukocyte and/or platelet activation. Nucleic acids and proteins which are capable of modulating leukocyte and/or platelet activation are provided. Compositions and methods for the treatment of disorders related to leukocyte and/or platelet activation are also provided. Prophylactics and methods for the prevention of such disorders are also provided. Also provided are compositions and methods for diagnostic and prognostic determination of such disorders. Further provided are assays for the identification of bioactive agents capable of modulating leukocyte and/or platelet activation.

In one aspect of the invention, nucleic acids and proteins capable of modulating leukocyte and/or platelet activation are provided. Accordingly, the present invention provides SLIM proteins, alternatively and equivalently referred to herein as SLAP-2 proteins, and SLIM nucleic acids, alternatively and equivalently referred to herein as SLAP-2 nucleic acids.

In a preferred embodiment, SLIM proteins and SLIM nucleic acids provided herein are capable of modulating leukocyte and/or platelet activation. In a further preferred embodiment, SLIM proteins and SLIM nucleic acids provided herein are capable of modulating antigen receptor-induced signaling and activation in leukocytes and/or platelets. In a further preferred embodiment, SLIM proteins and SLIM nucleic acids provided herein are capable of modulating antigen receptor-induced signaling and activation in lymphocytes and/or mast cells.

In a preferred embodiment, the SLIM nucleic add comprises a nucleic acid sequence having at least about 85%, more preferably at least about 90%, more preferably at least about 95%, more preferably at least about 98% identity to the nucleic acid sequence set forth in FIG. 2. In a preferred embodiment, the SLIM nucleic acid comprises the nucleic acid sequence set forth in FIG. 2. In a preferred embodiment, the SLIM nucleic acid encodes a SLIM protein.

In a preferred embodiment, the SLIM protein comprises an amino acid sequence encoded by a nucleic acid sequence having at least about 85%, more preferably at least about 90%, more preferably at least about 95%, more preferably at least about 98% identity to the nucleic acid sequence set forth in FIG. 2. In a preferred embodiment, the SLIM protein comprises an amino acid sequence encoded by the nucleic acid sequence set forth in FIG. 2.

Also provided herein are SLIM antisense nucleic acids which nucleic acids will hybridize under high stringency conditions to a SLIM nucleic acid comprising the nucleic acid sequence set forth in FIG. 2. In a preferred embodiment, antisense nucleic acid comprises a nucleic acid sequence complementary to that set forth in FIG. 2 or a fragment thereof. In a preferred embodiment, the SLIM antisense nucleic acid inhibits expression of SLIM protein encoded by SLIM nucleic acid. In a preferred embodiment, the SLIM antisense nucleic acid inhibits SLIM protein activity. In a preferred embodiment, the SLIM antisense nucleic acid has at least one activity possessed by a SLIM protein variant described herein.

In a preferred embodiment, the SLIM protein comprises an amino acid sequence having at least about 85%, more preferably at least about 90%, more preferably at least about 95%, more preferably at least about 98% identity to the amino acid sequence set forth in FIG. 2. In a preferred embodiment, the SLIM protein comprises the amino acid sequence set forth in FIG. 2. In a preferred embodiment, SLIM comprises an N-terminal SH2 domain, an N-terminal SH3 domain, an N-terminal myristylation sequence, and lacks a tyrosine kinase domain. Preferably, the SLIM protein also possesses one or more SLIM bioactivities described herein.

In a preferred embodiment, the SLIM protein binds to CbI protein. In a further preferred embodiment, SLIM protein binding to CbI is induced by antigen receptor activation.

In a preferred embodiment, the SLIM protein modulates antigen receptor activity. In a preferred embodiment, the SLIM protein binds to CbI protein which mediates the modulation of lymphocyte activity by antigen receptor.

In one aspect of the invention, expression vectors are provided. The expression vectors comprise one or more SLIM nucleic acids operably linked to regulatory sequences recognized by a host cell transformed with the expression vector. Further provided herein are host cells comprising expression vectors and SLIM nucleic acids. Also provided are processes for producing SLIM protein comprising culturing a host cell under conditions suitable for expression of the SLIM protein. In one embodiment, the process includes recovering the SLIM protein.

The present invention also provides isolated polypeptides which specifically bind to SLIM. In one aspect, the polypeptide is an antibody. In a preferred aspect, the polypeptide is a monoclonal antibody.

Also provided herein are methods for screening for bioactive agents capable of binding to SLIM. In a preferred embodiment, the methods comprise combining SLIM and a candidate bioactive agent and determining the binding of candidate agent to SLIM. In one aspect, the method involves identifying the candidate agent.

Also provided herein are methods for screening for a bioactive agent capable of modulating the binding of SLIM. In one embodiment, the methods comprise combining a candidate bioactive agent, SLIM protein, and a SLIM binding partner which will bind to SLIM in the absence of candidate agent and determining the binding of SLIM to binding partner in the presence of candidate bioactive agent. In a preferred aspect, the SLIM binding partner is CbI. The methods may additionally comprise determining the binding of SLIM to SLIM binding partner in the absence of candidate bioactive agent. The methods may additionally comprise identifying the candidate agent. In one embodiment, SLIM and SLIM binding partner are combined first. Candidate agents may increase, decrease or otherwise alter the binding of SLIM to SLIM binding partner.

Also provided herein are methods for screening for an agent capable of modulating the ubiquitination of a CbI target protein that is capable of being ubiquitinated. In one aspect, the method comprises combining SLIM, a candidate bioactive agent, ubiquitin or polyubiquitin, CbI and a CbI target protein and determining the level of ubiquitination of target protein in the presence of candidate agent. The methods may additionally comprise determining the level of ubiquitination in the absence of candidate agent. In a preferred embodiment, CbI target protein is a SLIM binding partner. In a further preferred embodiment, CbI target protein is additionally a component of an antigen receptor in lymphocytes and/or mast cells. In another preferred embodiment, CbI target protein is atyrosine kinase downstream of antigen receptor in lymphocytes and/or mast cells. The level of target protein ubiquitination may be determined by determining the amount of ubiquitin-conjugated target protein. Alternatively, the level of target protein ubiquitination may be determined by determining the amount of free ubiquitin.

In another embodiment, the methods comprise contacting a candidate agent with a cell comprising SLIM, CbI and CbI target protein, and determining the level of ubiquitination of CbI target protein in the presence and absence of candidate agent.

Also provided herein are methods for screening for a bioactive agent capable of modulating the activity of SLIM. In a preferred embodiment, the methods comprise contacting a candidate bioactive agent to a cell comprising recombinant SLIM nucleic acid and expressing SLIM protein. In another preferred embodiment, the methods comprise contacting a library of candidate bioactive agents to a plurality of cells comprising recombinant SLIM nucleic acid and expressing SLIM protein.

In one embodiment, the methods comprise expressing SLIM nucleic acid in a lymphocyte or mast cell, contacting the expressing cell with a candidate agent, and determining the level of expression of a surface marker which expression is normally correlated with lymphocyte or mast cell activation, in the presence of candidate agent. The methods may additionally comprise determining the level of surface marker expression in the absence of candidate agent. In a preferred embodiment, the surface marker is selected from the group consisting of CD23, CD69, CD80 and CD86. The surface marker CD69 is especially preferred.

In one embodiment, the methods comprise expressing SLIM nucleic acid in a lymphocyte or mast cell, contacting the expressing cell to a candidate agent, and determining the level of activity of a promoter which activity normally correlates with lymphocyte or mast cell activation, in the presence of candidate agent. The methods may additionally comprise determining the level of promoter activity in the absence of candidate agent. In a preferred embodiment the promoter is regulated by nuclear factor activated in T cells (NFAT) activity. In another preferred embodiment, the promoter is the immunoglobulin heavy chain gene promoter.

Also provided herein are methods for screening for a bioactive agent capable of modulating leukocyte and/or platelet activation. In one embodiment, the methods comprise contacting a candidate bioactive agent to a cell comprising recombinant SLIM nucleic acid and expressing SLIM protein, and determining the effect of candidate agent on the cell. In another embodiment, the methods comprise contacting a library of candidate bioactive agents to a plurality of cells comprising recombinant SLIM nucleic acid and expressing SLIM protein. In one embodiment, the methods comprise determining the level of ubiquitination of CbI target protein. In one embodiment, the methods comprise determining the binding of candidate agent to SLIM. In another embodiment, the methods comprise determining the modulation of SLIM binding by candidate agent. In another embodiment, the methods comprise determining the modulation of SLIM bioactivity by candidate agent.

Compositions and methods for the modulation of leukocyte and/or platelet activation are also provided herein. The compositions include proteins, nucleic acids and small molecule chemical compositions. In one embodiment, the compositions comprise SLIM proteins and/or SLIM nucleic acids. These compositions find use as prophylactics and therapeutics for the prevention and treatment of disorders associated with leukocyte and/or platelet activation, preferably disorders associated with SLIM bioactivity. In a preferred embodiment, compositions for the treatment of immunodeficiency disorders, such as AIDS, are provided. In another preferred embodiment, compositions useful for the prevention and treatment of acute inflammatory disorders, chronic inflammatory disorders, autoimmune disorders, and transplant rejection are provided.

### BRIEF DESCRIPTION OF THE DRAWINGS

FIG. 1 CD69 cell surface marker screen in BJAB cells

FIG. 1A Characterization of the tTA-BJAB cell line using dominant-negative DSyk. tTA-BJAB cell line was infected with pTRA-IRES.GFP vector or a truncated, dominant negative version of Syk (ΔSyk). Cells were stimulated with anti-IgM F(ab′)₂ and stained for surface CD69 expression. In GFP-gated control cells, 5 fold antigen-induced upregulation of CD69 expression was achieved over the basal level. Infection of ΔSyk reduced anti-IgM F(ab′)₂-induced CD69 expression to 38% of the control value and also slightly depressed basal CD69 expression. Geometric mean values of CD69 fluorescence are shown in brackets.

FIG. 1B Screening Strategy.

tTA BJAB cells stably infected with pTRA cDNA libraries were stimulated with anti-IgM F(ab′)₂ and stained for CD69 surface expression. Cells expressing the lowest levels of antigen-induced CD69 were sorted, expanded and resorted until significant enrichment of non-responsive cells was observed. Single cell clones were deposited from several sorts and analyzed for anti-IgM F(ab′)₂-induced CD69 upregulation in the absence (cDNA on) and presence (cDNA off) of doxicycline (dox).

FIG. 1C Cell surface CD69 expression in representative positive cell clones.

Unstimulated cells (dotted line) and cells stimulated with anti-IgM F(ab′)₂ (solid line) were stained with APC-conjugated anti-CD69 antibody. Grey line: stimulated cells stained with isotype control-APC antibody. Inhibiton of anti-IgMF(ab′)₂-induced CD69 expression (top panel) by cDNAs in clones 584, 780 and G18 (containing spiked control Asyk) is reversed when cells are grown in the presence of dox (cDNA off; lower panel).

FIG. 2A Human SLIM cDNA and protein sequences. The putative myristylation site is shown in bold. Open and solid triangles indicate the boundaries of SH3 and SH2 domains, respectively. These sequence data are available at Genbank under accession no. AF326353.

FIG. 2B Alignment of human SLIM and SLAP

Identical amino acids are boxed and highlighted. Open and solid triangles indicate the boundaries of SH3 and SH2 domains, respectively. The overall amino acid similarity between SLIM and SLAP is 36%. In the SH2 and SH3 domains alone, the similarity between SLIM and SLAP is 48% and between SLIM and the Src family kinase Hck is 45%.

FIG. 2C Northern blot analysis of SLIM expression

Left: human tissues and right: tumor cell lines including promyelocytic leukemia HL-60, HeLa cell S3, chronic myelogeneous leukemia K-562, lymphoblastic leukemia MOLT-4, Burkitt's lymphoma Raji, colorectal adenocarcinoma SW480, lung carcinoma A549, and melanoma G361. Blots were stripped and reprobed with an actin probe (bottom panel).

FIG. 2D RT-PCR of SLIM from purified human primary cells

Specific SLIM primers or control GAPDH primers were used in a standard PCR reaction with cDNA templates obtained from resting and activated CD4+ T cells (stimulated with PHA), CD19+ B cells (stimulated with pokeweed mitogen), or CD14+ (human blood fractions MTC panel (Clontech)).

FIG. 3A Inhibition of CD69 upregulation by SLIM and SLAP in tTA-BJAB cells

Epitope-tagged SLIM and SLAP cDNAs in the pTRA-IRES.GFP vector were infected into naive tTA-BJAB cells. Surface CD69 expression was analyzed in unstimulated (dotted line) and anti-IgM F(ab′)₂-stimulated (solid line) cells. The ratio of the induced and uninduced geometric mean of APC-CD69 fluorescence is shown for uninfected (GFP negative) and infected (GFP positive) cells. Both SLIM and SLAP decrease anti-IgM F(ab′)₂-induced CD69 upregulation to approximately 50% of control values.

FIG. 3B Anti-CD3 induced CD69 expression is inhibited by SLIM

Jurkat-N cells were transiently transfected with SLIM in pEFBOS-IRES.GFP or vector alone. Cells were stimulated with anti-CD3 Leu4 (1 μg/ml) or PMA (50 ng/ml) for 24 hrs and stained for CD69-PE. CD69 expression in GFP positive cells is represented graphically (unstimulated=line; stimulated solid) and the geometric mean values (fluorescence intensity) are summarized in the table.

FIG. 3C Anti-CD3-induced NFAT promoter activation is inhibited by SLIM

Jurkat-N cells were transiently co-transfected with SLIM in pEFBOS-IRES.GFP or vector alone, and an NFAT-Luciferase reporter construct. Cells were stimulated with anti-CD3, or PMA plus ionomycin for 12 hrs, and assayed for luciferase activity. Fold induction of luciferase activity over the unstimulated, vector-transfected sample is shown. The basal luciferase activity for this experiment was approximately 100 arbitrary light units (AU). The data are representative of two independent experiments.

FIG. 4 The N-terminal myristylation site and C-terminal unique regions of SLIM are required for inhibition of antigen receptor signaling

Epitope-tagged wild type SLIM, SLIM-myr and SLIM-AC in the pTRA-IRES.GFP vector were stably introduced into tTA-BJAB cells, and GFP-positive cells were purified by sorting. Cells were stimulated with anti-IgM F(ab′)₂ and stained for surface expression of CD69. Wild type SLIM decreased CD69 induction to 53% of the control value, whereas SLIM-myr and SLIM-AC resembled the vector control. The ratio of the induced and uninduced geometric means is shown.

FIG. 5A SLIM associates with tyrosine phosphorylated proteins in B cells Sorted tTA-BJAB cells infected with epitope-tagged wild type SLIM, SLIM-myr or SLIM-ΔC were stimulated with anti-IgM F(ab′)₂ for 2 mins, lysed and SLIM was immunoprecipitated using anti-FLAG antibodies. Immunoprecipitated proteins were subjected to SDS-PAGE and immunoblotted with anti-phosphotyrosine antibodies. SLIM associates with tyrosine phosphorylated proteins of approximately 110 and 70 kDa after antigen stimulation. The ΔC mutant lacks the 110 kDa SLIM-associated phosphoprotein. Lower panel: Reprobe with anti-FLAG.

FIG. 5B SLIM interacts with CbI in B cells

Wild type SLIM, SLIM-myr or SLIM-ΔC were immunoprecipitated as in A and immunoblotted with anti-CbI antibodies. WT SLIM and SLIM-myr but not SLIM-DC associate with CbI after antigen stimulation.

FIG. 6 SLAP-2 (SLIM) inhibits antigen receptor signaling in B and T lymphocytes

Epitope-tagged SLAP-2 and SLAP cDNAs in the pTRA-IRES.GFP vector or vector alone were infected into tTA-BJAB or Jurkat cells. Surface CD69 expression was analyzed in stimulated GFP-negative (uninfected; dotted lone) and GFP-positive (infected; solid line) cells by analytical gating. The geometric means of APC-CD69 fluorescence are shown for GFP-negative and GFP-positive cells. (A) tTA-BJAB cells stimulated with anti-IgM F(ab′)₂ or PMA. Both SLAP-2 and SLAP significantly decrease anti-Igm F(ab′)₂-induced but not PMA-induced CD69 expression compared with control. (B) tTA-Jurkat cells stimulated with C305 or PMA. Both SLAP-2 and SLAP significantly decrease C305-induced CD69 expression compared with control. (C) NFAT promoter activation is inhibited by SLAP-2. BJAB or Jurkat-TAg cells were transiently co-transfected with 40 p.g pEFBOS-SLAP-2 or vector alone, plus an NFAT-Luciferase reporter construct. Cells were stimulated with anti-Igm F(ab′)₂, C305, or PMA plus ionomycin for 12 h, and assayed for luciferase activity in triplicate. Fold induction of luciferase activity over the unstimulated, vector transfected sample is shown. The basal luciferase activity for this experiment was −100 arbitrary light units (AU). The data are representative of several independent experiments. (D) Jurkat-TAg cells were transiently cotransfected with 1, 2, 4, 16 or 32 p.g of SLAP-2 DNA and FNFAT-Luciferase reporter construct, keeping the total DNA amount constant with empty vector. Cells were stimulated and luciferase assays were performed as above. Equal aliquots of cells were analyzed by anti-FLAG Westem Blot for SLAP-2 protein expression.

FIG. 7 SLAP-2 represses antigen-induced calcium mobilization in BJAB and Jurkat cells.

BJAB and Jurkat cells stably expressing SLAP-2 were labeled with the calcium indicator indo-1. Cells were stimulated at 37° C. on an LSR flow cytometer with UV laser, and analyzed by analytical gating on GFP. Ration of bound/unbound indo-1 is shown over a 10-min time course. Broken line: GFP-negative cells; solid line: GFP-positive cells. (A) BJAB cells stimulated with anti-Igm F(ab′)₂ followed by ionomycin. (B) Jurkat cells stimulated with C305 followed by ionomycin.

FIG. 8 The N-terminal myristoylation site and C-terminal unique regions of SLAP-2 are required for inhibition of antigen receptor signaling.

Epitope-tagged wild-type SLAP-2, SLAP-2-myr, and SLAP-2-ΔC in the pTRA-IRES.GFP vector were stably introduced into tTA-BJAB or tTA-Jurkat cells, and GFP-positive cells were enriched by sorting. Induced surface CD69 expression was analyzed in vector-infected cells (dotted line) and cells infected with wild-type or mutant SLAP-2 (solid line) with analytical-gating on GFP expression. The geometric means of APC-CD69 Fluorescence is shown. (A) tTA-BJAB cells stimulated with anti-Igm F(ab′)₂ Wild-Type (WT) SLAP-2 reduced anti-Igm F(ab′)₂-induced DC69 expression by ˜2 fold whereas SLAP-2-myr and SLAP-2ΔC resembled the vector control. Percentage of GFP positive cells: vector 91% wild-type SLAP-2 89%, SLAP-2-myr 80%, SLAP-2-C 87%. (B) tTA-Jurkat cells stimulated with C305. Wild-type SLAP-2 reduced C305-induced CD69 expression by ˜3 fold, whereas inhibition was compromised in cells expressing SLAP-2-myr and SLAP-2-ΔC. Percentage of GFP-positive cells: vector 84%, wild-type SLAP-2 46%; SLAP-2-myr 44%, SLAP-2-ΔC 45%. (C) Equal aliquots of infected tTA-BJAB cells were lysed and analyzed by anti-FLAG Western blot for SLAP-2 protein expression. (D) Equal aliquots of infected tTA-Jurkat cells were lysed and analyzed by anti-FLAG Western blot for SLAP-2 protein expression. (E) Membrane pellet (P) and soluble fraction (S) of wild-type SLAP-2 and SLAP-2-myr-infected tTA-BJAB cells were immunoprecipitated (top panel) or loaded directly (center bottom panels) and immunoblotted with antibodies raised against the FLAG epitope (top panel), the integral membrane protein CD40 (center panel), and the cytoplasmic protein JNK (bottom panel). A fraction of wild-type SLAP-2 but not SLAP-2-myr was localized in the membrane fraction.

FIG. 9 SLAP-2 associates with tyrosine phosphorylated proteins including CbI after antigen stimulation.

FIG. 9A SLAP-2 associates with tyrosine phosphorylated proteins in B cells. Sorted tTA-BJAB cells infected with epitope-tagged wildtype (WT) SLAP-2, SLAP-2-myr, or SLAP-2-ΔC were stimulated with anti-Igm F9ab′)2 for 2 min, lysed, and SLAP-2 was immunoprecipitated using antiFLAG agarose. Immunopreceipitated proteins were subjected to SDS-PAGE and immunoblotted with anti-phosphotyrosine antibodies. SLAP-2 associates with tyrosine phosphorylated proteins of ˜110and 70 kD after antigen stimulation. The ΔC mutant lacks the 110 kD SLAP-2-associated phosphoprotein. Bottom panel: reprobe with anti-FLAG.

FIG. 9B SLAP-2 interacts with CbI in B cells. Wildtype SLAP-2, SLAP-2-myr, or SLAP-2-ΔC were immunoprecipitated as in (A) and immunoblotted with anti-CbI antibodies. Wildtype SLAP-2 and SLAP-2-myr but not SLAP-2-ΔC associate with CbI after antigen stimulation.

### DETAILED DESCRIPTION OF THE INVENTION

The present invention provides compositions and methods for modulating leukocyte and/or platelet activation. Nucleic acids and proteins which are capable of modulating leukocyte and/or platelet activation are provided. Compositions and methods for the treatment of disorders related to leukocyte and/or platelet activation are also provided. Prophylactics and methods for the prevention of such disorders are also provided. Also provided are compositions and methods for diagnostic and prognostic determination of such disorders. Further provided are assays for the identification of bioactive agents capable of modulating leukocyte and/or platelet activation.

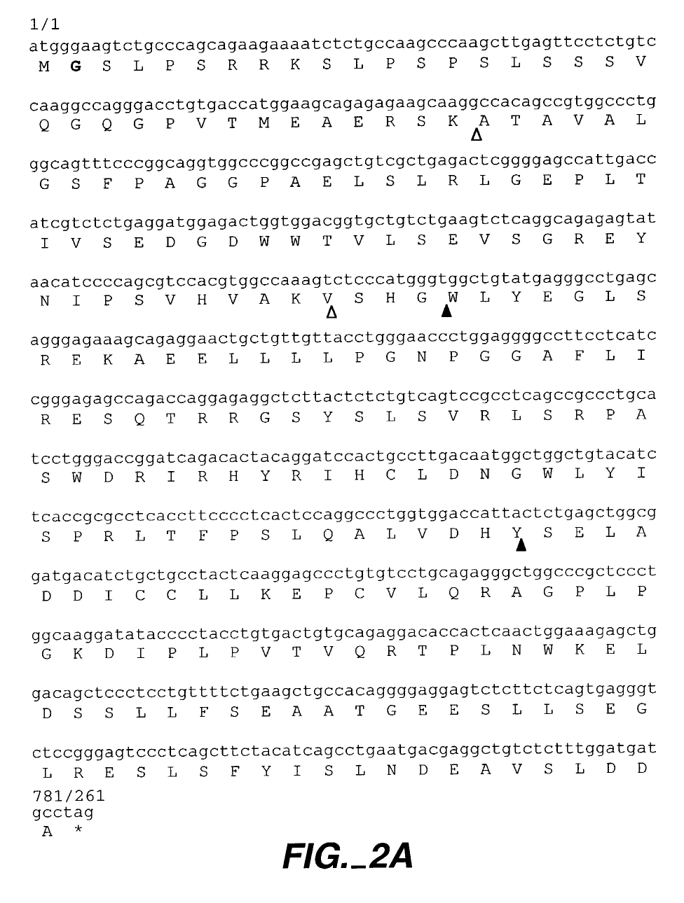

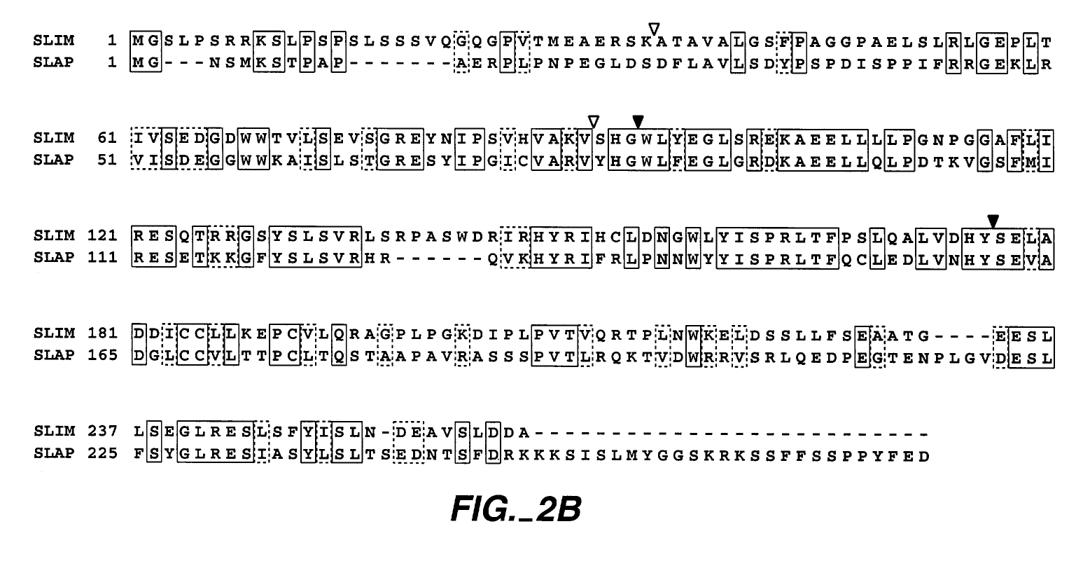

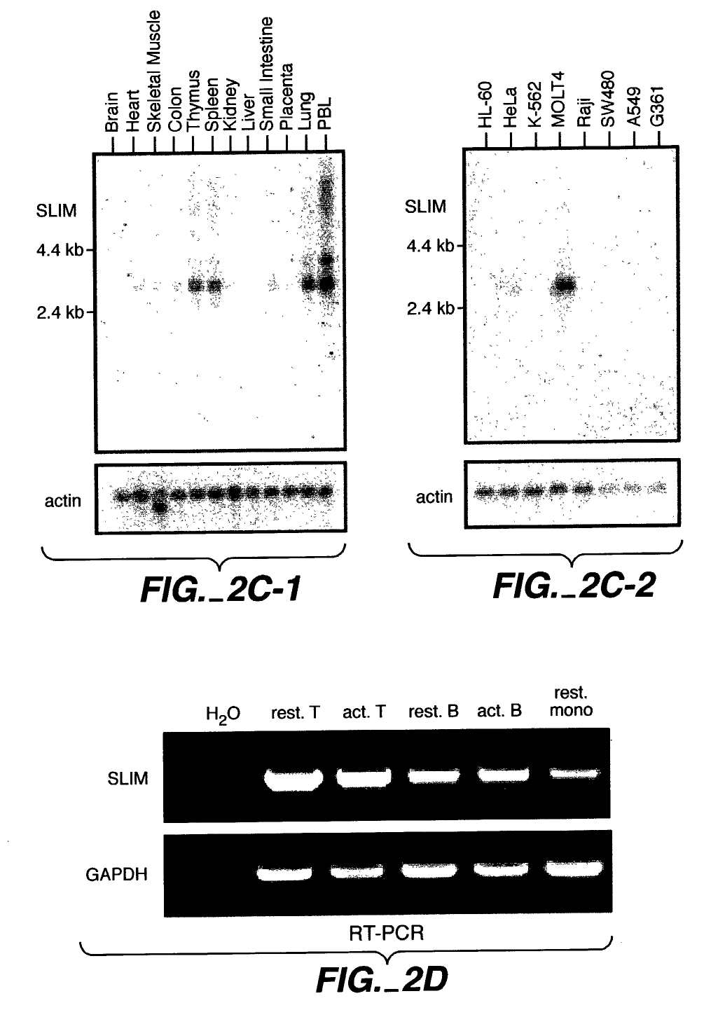

In one aspect of the invention, nucleic acids and proteins capable of modulating leukocyte and/or platelet activation are provided. Accordingly, the present invention provides SLIM proteins, alternatively and equivalently referred to herein as SLAP-2 proteins, and SLIM nucleic acids, alternatively and equivalently referred to herein as SLAP-2 nucleic acids. In one embodiment, the present invention provides SLIM nucleic acids comprising the nucleic acid sequence set forth in FIG. 2.

In one embodiment, the present invention provides SLIM proteins comprising the amino acid sequence set forth in FIG. 2.

In a preferred embodiment, SLIM proteins and SLIM nucleic acids provided herein are capable of modulating leukocyte and/or platelet activation. In a further preferred embodiment, SLIM proteins and SLIM nucleic acids provided herein are capable of modulating antigen receptor-induced signaling and activation in leukocytes and/or platelets. In a further preferred embodiment, SLIM proteins and SLIM nucleic acids provided herein are capable of modulating antigen receptor-induced signaling and activation in lymphocytes and/or mast cells.

A SLIM protein of the present invention may be identified in several ways. “Protein” in this sense includes proteins, polypeptides, and peptides. A SLIM nucleic acid or SLIM protein may be initially identified by substantial nucleic acid and/or amino acid sequence homology to the sequences shown in FIG. 2. Such homology can be based upon the overall nucleic acid or amino acid sequence. In addition, preferred SLIM proteins of the present invention comprise an SH2 domain, an SH3 domain, a myristylation sequence, and lack a tyrosine kinase domain. Preferred SLIM proteins of the present invention also possess SLIM protein activity as described below. SLIM proteins that bind to CbI are especially preferred. In a preferred embodiment, SLIM proteins bind to CbI in response to activation of antigen receptor.

CbI is a RING finger ubiquitin ligase protein which has been shown to be a negative regulator of TCR and BCR signaling pathways (Rudd et. al., Curr. Biol. 10:R344-R347, 2000; Ota et. al., J. Biol. Chem. 275:414-422, 2000; Rao et. al., J. Immunol., 164:4616-4626, 2000; Holland et. al., J, Exp. Med., 194:1263-1276, 2001).

As used herein, a protein is a “SLIM protein” if the overall homology of the protein sequence to the amino acid sequence shown in FIG. 2 is preferably greater than about 75%, more preferably greater than about 80%, even more preferably greater than about 85% and most preferably greater than 90%. In some embodiments the homology will be as high as about 93 to 95 or 98%. Homology in this context means sequence similarity or identity, with identity being preferred. This homology will be determined using standard techniques known in the art, including, but not limited to, the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et. al., Nucl. Acid Res. 12:387-395 (1984), preferably using the default settings, or by inspection.

One example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

Another example of a useful algorithm is the BLAST algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Altschul et al., Methods in Enzymology, 266: 460-480 (1996);]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, “percent (%) nucleic acid sequence identity” with respect to the coding sequence of the polypeptides identified herein is defined as the percentage of nucleotide residues in a candidate sequence that are identical with the nucleotide residues in the coding sequence of the SLIM protein. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer amino acids than the protein shown in FIG. 2, it is understood that the percentage of homology will be determined based on the number of homologous amino acids in relation to the total number of amino acids. Thus, for example, homology of sequences shorter than that shown in the Figure, as discussed below, will be determined using the number of amino acids in the shorter sequence.

SLIM proteins of the present invention have structural homology to the known SLAP protein but are distinct therefrom. Comparison of the amino acid sequence of a preferred SLIM protein, which amino acid sequence is set forth in FIG. 2, and the amino acid sequence of human SLAP, set forth at Genbank accession number NM_{—}006748, reveals an overall sequence similarity of 36% and less than 50% similarity through the shared SH2 and SH3 domains (see FIG. 2 and Holland et. al., J. Exp. Med. 194:1263-1276, 2001). The C-terminal sequences of SLIM and SLAP proteins are divergent. Notably, it is the C-terminal domain of SLIM that mediates CbI binding.

SLIM proteins of the present invention may be shorter or longer than the amino acid sequences shown in the Figure. Thus, in a preferred embodiment, included within the definition of SLIM proteins are portions or fragments of the sequences depicted herein. Fragments of SLIM proteins are considered SLIM proteins if a) they share at least one antigenic epitope; b) have at least the indicated homology; c) and preferably have SLIM biological activity, which may include the ability to bind CbI and/or the ability to modulate antigen receptor-induced lymphocyte activation. However, variant SLIM proteins which lack one or more SLIM activities (sometimes referred to herein as SLIM variants), as further described below, are also considered SLIM proteins for the purposes of the present invention.

In a preferred embodiment, the SLIM proteins are derivative or variant SLIM proteins. That is, as outlined more fully below, the derivative SLIM peptide will contain at least one amino acid substitution, deletion or insertion, with amino acid substitutions being particularly preferred. The amino acid substitution, insertion or deletion may occur at any residue within the SLIM peptide.

In addition, as is more fully outlined below, SLIM proteins can be made that are longer than those depicted in the figures, for example, by the addition of epitope or purification tags, the addition of other fusion sequences including fusions to reporter genes or proteins such as green fluorescent protein (GFP, including both Aqueorea species and Renilla species), etc.

SLIM proteins may also be identified as being encoded by SLIM nucleic acids. Thus, SLIM proteins are encoded by nucleic acids that will hybridize to the sequence depicted in FIG. 2, or its complement, as outlined herein. In a preferred embodiment, SLIM proteins provided herein are encoded by a SLIM nucleic acid comprising a nucleic acid sequence having at least about 75%, more preferably at least about 80%, more preferably at least about 85%, more preferably at least about 90%, more preferably at least about 95%, more preferably at least about 98% identity to the nucleic acid sequence set forth in FIG. 2.

In a preferred embodiment, when the SLIM protein is to be used to generate antibodies, the SLIM protein must share at least one epitope or determinant with the full length protein shown in FIG. 2. By “epitope” or “determinant” herein is meant a portion of a protein which will generate and/or bind an antibody. Thus, in most instances, antibodies made to a smaller SLIM protein will be able to bind to the full length protein. In a preferred embodiment, the epitope is unique; that is, antibodies generated to a unique epitope show little or no cross-reactivity. The term “antibody” includes antibody fragments, as are known in the art, including Fab Fab₂, single chain antibodies (Fv for example), chimeric antibodies, etc., either produced by the modification of whole antibodies or those synthesized de novo using recombinant DNA technologies.

In a preferred embodiment, the antibodies to SLIM are capable of reducing or eliminating the biological function of SLIM, as is described herein. That is, the addition of anti-SLIM antibodies (either polyclonal or preferably monoclonal) to SLIM (or cells containing SLIM) may reduce or eliminate the SLIM activity. Generally, at least a 25% decrease in activity is preferred, with at least about 50% being particularly preferred and about a 95-100% decrease being especially preferred.

The SLIM antibodies of the invention specifically bind to SLIM proteins. By “specifically bind” herein is meant that the antibodies bind to the protein with a binding constant in the range of at least 10⁻⁴-10⁻⁶ M⁻¹, with a preferred range being 10⁻⁷-10⁻⁹ M⁻¹.

In the case of the nucleic acid, the overall homology of the nucleic acid sequence is commensurate with amino acid homology but takes into account the degeneracy in the genetic code and codon bias of different organisms. Accordingly, the nucleic acid sequence homology may be either lower or higher than that of the protein sequence. Thus the homology of the nucleic acid sequence as compared to the nucleic acid sequence of FIG. 2 is preferably greater than 75%, more preferably greater than about 80%, particularly greater than about 85% and most preferably greater than 90%. In some embodiments the homology will be as high as about 93 to 95 or 98%.

In a preferred embodiment, a SLIM nucleic acid encodes a SLIM protein. As will be appreciated by those in the art, due to the degeneracy of the genetic code, an extremely large number of nucleic acids may be made, all of which encode the SLIM proteins of the present invention. Thus, having identified a particular amino acid sequence, those skilled in the art could make any number of different nucleic acids, by simply modifying the sequence of one or more codons in a way which does not change the amino acid sequence of the SLIM protein.

In one embodiment, the nucleic acid homology is determined through hybridization studies. Thus, for example, nucleic acids which hybridize under high stringency to the nucleic acid sequences shown in FIG. 2 or its complement is considered a SLIM gene. High stringency conditions are known in the art; see for example Maniatis et al., Molecular Cloning: A Laboratory Manual, 2d Edition, 1989, and Current Protocols in Molecular Biology, ed. Ausubel, et al., J. Wiley & Sons publ., New York, 1988, both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength and pH. The Tₘ is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as form amide.

In another embodiment, less stringent hybridization conditions are used; for example, moderate or low stringency conditions may be used, as are known in the art; see Maniatis and Ausubel, supra, and Tijssen, supra.

The SLIM proteins and SLIM nucleic acids of the present invention are preferably recombinant. As used herein, “nucleic acid” may refer to either DNA or RNA, or molecules which contain both deoxy- and ribonucleotides. The nucleic acids include genomic DNA, cDNA and oligonucleotides including sense and anti-sense nucleic acids. In one embodiment, SLIM nucleic acids are antisense nucleic acids capable of inhibiting expression of SLIM protein encoded by a SLIM nucleic acid. Nucleic acids of the present invention may also contain modifications in the ribose-phosphate backbone to increase stability and half life of such molecules in physiological environments.

The nucleic acid may be double stranded, single stranded, or contain portions of both double stranded or single stranded sequence. As will be appreciated by those in the art, the depiction of a single strand (“Watson”) also defines the sequence of the other strand (“Crick”); thus the sequence depicted in FIG. 2 also includes the complement of the sequence. By the term “recombinant nucleic acid” herein is meant nucleic acid, originally formed in vitro, in general, by the manipulation of nucleic acid by endonucleases, in a form not normally found in nature. Thus an isolated SLIM nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a SLIM protein from one organism in a different organism or host cell. Alternatively, the protein may be made at a significantly higher concentration than is normally seen, through the use of a inducible promoter or high expression promoter, such that the protein is made at increased concentration levels. Alternatively, the protein may be in a form not normally found in nature, as in the addition of an epitope tag or amino acid substitutions, insertions and deletions, as discussed below.

Also included within the definition of SLIM proteins of the present invention are amino acid sequence variants. These variants fall into one or more of three classes: substitutional, insertional or deletional variants. These variants ordinarily are prepared by site specific mutagenesis of nucleotides in the DNA encoding the SLIM protein, using cassette or PCR mutagenesis or other techniques well known in the art, to produce DNA encoding the variant, and thereafter expressing the DNA in recombinant cell culture as outlined above. However, variant SLIM protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the SLIM protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed SLIM variants screened for the optimal combination of desired activity. Techniques for making substitution mutations at predetermined sites in DNA having a known sequence are well known, for example, M13 primer mutagenesis and PCR mutagenesis. Screening of the mutants is done using assays of SLIM protein activities.

Amino acid substitutions are typically of single residues; insertions usually will be on the order of from about 1 to about 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the SLIM protein are desired, substitutions are generally made in accordance with the following chart:

CHART I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the SLIM proteins as needed. Alternatively, the variant may be designed such that the biological activity of the SLIM protein is altered.

Accordingly, in one aspect of the invention, SLIM variants that lack at least one SLIM bioactivity are provided. In a preferred embodiment, SLIM variants are unable to bind to CbI. Such SLIM variants include SLIM proteins which lack the C-terminal domain required for binding to CbI and for inhibition of antigen receptor-induced lymphocyte activation. In a preferred embodiment, such SLIM variants comprise amino acids 1-194 depicted FIG. 2. in a preferred embodiment, such SLIM variants lack the amino acid sequence set forth by amino acids 195-261 in FIG. 2. In another preferred embodiment, SLIM variants that do not localize to the plasma membrane are provided. Such SLIM variants include SLIM proteins that lack the N-terminal myristylation sequence that facilitates membrane localization and inhibition of antigen receptor-induced lymphocyte activation by SLIM protein. In a preferred embodiment, such a SLIM variant has a point mutation at the second amino acid residue (Gly) of the amino acid sequence set forth in FIG. 2. In another preferred embodiment, SLIM variants that are unable to bind to tyrosine phosphorylated SLIM binding partners, which include tyrosine kinases and phosphatases that are modulated by antigen receptor activation, are provided. Such SLIM variants include SLIM proteins that lack an N-terminal SH2 domain. In another preferred embodiment, SLIM variants that are unable to bind to SLIM binding partners comprising proline rich regions are provided. Such SLIM variants include SLIM proteins that lack an N-terminal SH3 domain.

In a preferred embodiment, SLIM variants possess dominant negative activity. That is, such SLIM variants are able to inhibit at least one bioactivity of non-variant SLIM in trans.

In one embodiment, SLIM variant proteins inhibit the ability of SLIM to inhibit antigen receptor-induced lymphocyte activation. Such SLIM variants can increase antigen receptor-induced lymphocyte activation.

In one embodiment, SLIM variant proteins bind to CbI but do not localize to the plasma membrane. Such SLIM variants can increase antigen receptor-induced lymphocyte activation. Such SLIM variants inclide SLIM proteins lacking an N-terminal myristylation sequence.

In one embodiment, SLIM variant proteins interact with SLIM binding partners comprising phosphotyrosine or proline rich regions, but do not interact with CbI. Such SLIM variants can increase antigen receptor-induced lymphocyte activation.

In addition, SLIM variants that can increase lymphocyte activation from rest (i.e. in the absence of lymphocyte activation, for example antigen receptor-induced lymphocyte activation, are provided herein.

Determining whether SLIM variant has dominant negative activity can be done using the assays provided herein, particularly assays provided for screening for bioactive agents capable of modulating lymphocyte activation. Especially preferred are assays that involve measuring endogenous markers or activities or surrogate markers or activities that are indicators of lymphocyte activation, such as appropriate surface molecule expression or appropriate promoter activity.

Covalent modifications of SLIM polypeptides are included within the scope of this invention. One type of covalent modification includes reacting targeted amino acid residues of a SLIM polypeptide with an organic derivatizing agent that is capable of reacting with selected side chains or the N-or C-terminal residues of a SLIM polypeptide. Derivatization with bifunctional agents is useful, for instance, for crosslinking SLIM to a water-insoluble support matrix or surface for use in the method for purifying anti-SLIM antibodies or screening assays, as is more fully described below. Commonly used crosslinking agents include, e.g., 1,1-bis(diazoacetyl)-2-phenylethane, glutaraldehyde, N-hydroxy-succinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the “-amino groups of lysine, arginine, and histidine side chains [T.E. Creighton, Proteins: Structure and Molecular Properties, W.H. Freeman & Co., San Francisco, pp. 79-86 (1983)], acetylation of the N-terminal amine, amidation of any C-terminal carboxyl group, and sulfation of side chains.

Another type of covalent modification of the SLIM polypeptide included within the scope of this invention comprises altering the native glycosylation pattern of the polypeptide. “Altering the native glycosylation paftern” is intended for purposes herein to mean deleting one or more carbohydrate moieties found in native sequence SLIM polypeptide, and/or adding one or more glycosylation sites that are not present in the native sequence SLIM polypeptide.

Addition of glycosylation sites to SLIM polypeptides may be accomplished by altering the amino acid sequence thereof. The alteration may be made, for example, by the addition of, or substitution by, one or more serine or threonine residues to the native sequence SLIM polypeptide (for O-linked glycosylation sites). The SLIM amino acid sequence may optionally be altered through changes at the DNA level, particularly by mutating the DNA encoding the SLIM polypeptide at preselected bases such that codons are generated that will translate into the desired amino acids.

Another means of increasing the number of carbohydrate moieties on the SLIM polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the SLIM polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of SLIM comprises linking the SLIM polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

SLIM polypeptides of the present invention may also be modified in a way to form chimeric molecules comprising a SLIM polypeptide fused to another, heterologous polypeptide or amino acid sequence. In one embodiment, such a chimeric molecule comprises a fusion of a SLIM polypeptide with a tag polypeptide which provides an epitope to which an anti-tag antibody can selectively bind. The epitope tag is generally placed at the amino-or carboxyl-terminus of the SLIM polypeptide. The presence of such epitope-tagged forms of a SLIM polypeptide can be detected using an antibody against the tag polypeptide. Also, provision of the epitope tag enables the SLIM polypeptide to be readily purified by affinity purification using an anti-tag antibody or another type of affinity matrix that binds to the epitope tag. In an alternative embodiment, the chimeric molecule may comprise a fusion of a SLIM polypeptide with a reporter protein, an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Nat[. Acad. Sci. USA, 87:6393-6397 (1990)].

Also included with the definition of SLIM protein are other SLIM proteins of the SLIM family, and SLIM proteins from other organisms, which are cloned and expressed as outlined below. Thus, probe or degenerate polymerase chain reaction (PCR) primer sequences may be used to find other related SLIM proteins from humans or other organisms. As will be appreciated by those in the art, particularly useful probe and/or PCR primer sequences include the unique areas of the SLIM nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art.

Once the SLIM nucleic acid is identified, it can be cloned and, if necessary, its constituent parts recombined to form the entire SLIM nucleic acid. Once isolated from its natural source, e.g., contained within a plasmid or other vector or excised therefrom as a linear nucleic acid segment, the recombinant SLIM nucleic acid can be further-used as a probe to identify and isolate other SLIM nucleic acids. It can also be used as a “precursor” nucleic acid to make modified or variant SLIM nucleic acids and proteins.

Using the nucleic acids of the present invention which encode a SLIM protein, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the SLIM protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in the same reading frame. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the SLIM protein; for example, transcriptional and translational regulatory nucleic acid sequences from Bacillus are preferably used to express the SLIM protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention. Particularly preferred inducible promoters are tet-inducible promoters known in the art. These promoters provide for the induction (tet-on), or alternatively the repression (tet-off) of transcription in the presence of tetracycline or doxycycline.

A preferred expression vector system is a retroviral vector system such as is generally described in PCT/US97/01019 and PCT/US97/01048, both of which are hereby expressly incorporated by reference.

In a preferred embodiment, SLIM nucleic acids of the present invention are single stranded RNA nucleic acids which are present in the retroviral genome of an infectious retroviral particle. Particularly preferred are retroviral particles which are capable of only a single infection which are unable to replicate and direct their own packaging in cis in an infected host cell. Retroviral vectors known in the art may be used in conjunction with packaging cell lines known in the art to produce such infectious retroviral particles that are limited to a single infection (for example see U.S. Pat. No. 6,153,380 issued to Nolan et. al., Nov. 28, 2000). Cloning SLIM nucleic acids into such retroviral vectors and using the vectors to produce infectious retroviral particles produces SLIM nucleic acids that are single stranded RNA nucleic acids contained in a modified retroviral genome. Retroviral infectious particles are particularly useful for the introduction of heterologous nucleic acids to host cells by transduction, as is well known in the art.

Thus, in one embodiment, the present invention provides retroviral vectors comprising SLIM nucleic acids. In another embodiment, methods for producing retroviral particles comprising SLIM nucleic acids are provided. In another embodiment, methods for introducing SLIM nucleic acids to host cells by transduction are provided. In a preferred embodiment, SLIM nucleic acids are operably linked to regulatory sequences that provide for the expression of SLIM protein in transduced host cells.

The SLIM proteins of the present invention are produced by culturing a host cell transformed with an expression vector containing nucleic acid encoding a SLIM protein, under the appropriate conditions to induce or cause expression of the SLIM protein. The conditions appropriate for SLIM protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archebacteria, fungi, and insect and animal cells, including mammalian cells. Of particular interest are Drosophila melangaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, immortalized mammalian myeloid and lymphoid cell lines, Jurkat cells, mast cells and other endocrine and exocrine cells, and neuronal cells.

The SLIM protein may also be made as a fusion protein, using techniques well known in the art. Thus, for example, for the creation of monoclonal antibodies, if the desired epitope is small, the SLIM protein may be fused to a carrier protein to form an immunogen. Alternatively, the SLIM protein may be made as a fusion protein to increase expression, or for other reasons. For example, when the SLIM protein is a SLIM peptide, the nucleic acid encoding the peptide may be linked to other nucleic acid for expression purposes. Similarly, SLIM proteins of the invention can be linked to protein labels, such as green fluorescent protein (GFP), red fluorescent protein (RFP), blue fluorescent protein (BFP), yellow fluorescent protein (YFP), etc.

In one embodiment, the SLIM nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes, including fluorescent proteins such as GFP. The labels may be incorporated into the compound at any position.

In a preferred embodiment, the SLIM protein is purified or isolated after expression. SLIM proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the SLIM protein may be purified using a standard anti-SLIM antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the SLIM protein. In some instances no purification will be necessary.

Once expressed and purified if necessary, the SLIM proteins and nucleic acids are useful in a number of applications as outlined herein.

The nucleotide sequences (or their complement) encoding SLIM proteins have various applications in the art of molecular biology, including uses as hybridization probes, in chromosome and gene mapping and in the generation of anti-sense RNA and DNA. SLIM protein nucleic acid will also be useful for the preparation of SLIM proteins by the recombinant techniques described herein.

The full-length native sequence SLIM gene, or portions thereof, may be used as hybridization probes for a cDNA library to isolate other genes (for instance, those encoding naturally-occurring variants of SLIM or SLIM from other species) which have a desired sequence identity to the SLIM coding sequence. Optionally, the length of the probes will be about 20 to about 50 bases. The hybridization probes may be derived from the nucleotide sequences herein or from genomic sequences including promoters, enhancer elements and introns of native sequences as provided herein. By way of example, a screening method will comprise isolating the coding region of the SLIM gene using the known DNA sequence to synthesize a selected probe of about 40 bases. Hybridization probes may be labeled by a variety of labels, including radionucleotides such as ³²P or ³S, or enzymatic labels such as alkaline phosphatase coupled to the probe via avidin/biotin coupling systems. Labeled probes having a sequence complementary to that of the SLIM gene of the present invention can be used to screen libraries of human cDNA, genomic DNA or mRNA to determine which members of such libraries the probe hybridizes.

Nucleotide sequences encoding SLIM protein can also be used to construct hybridization probes for mapping the gene which encodes SLIM protein and for the genetic analysis of individuals. The nucleotide sequences provided herein may be mapped to a chromosome and specific regions of a chromosome using known techniques, such as fluorescence in situ hybridization particularly of metaphase chromosomes, linkage analysis against known chromosomal markers, and hybridization screening with libraries.

Nucleic acids which encode SLIM protein can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding a SLIM protein can be used to clone genomic DNA encoding a SLIM protein in accordance with established techniques and the genomic sequences used to generate transgenic animals that contain cells which express the desired DNA. Methods for generating transgenic animals, particularly animals such as mice or rats, have become conventional in the art and are described, for example, in U.S. Pat. Nos. 4,736,866 and 4,870,009. Typically, particular cells would be targeted for the SLIM protein transgene incorporation with tissue-specific enhancers. Transgenic animals that include a copy of a transgene encoding a SLIM protein introduced into the germ line of the animal at an embryonic stage can be used to examine the effect of increased expression of the desired nucleic acid. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of the SLIM protein can be used to construct a SLIM “knock out” animal which has a defective or altered SLIM gene as a result of homologous recombination between the endogenous gene encoding a SLIM protein and altered genomic DNA introduced into an embryonic cell of the animal. For example, cDNA encoding a SLIM protein can be used to clone genomic DNA encoding a SLIM protein in accordance with established techniques. A portion of the genomic DNA encoding a SLIM protein can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a ‘knock out’ animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the SLIM protein.

It is understood that the models described herein can be varied. For example, “knock-in” models can be formed, or the models can be cell-based rather than animal models.

Nucleic acids encoding SLIM polypeptides or antagonists or agonists of SLIM bioactivity may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83, 4143-4146 [1986]). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 [1993]). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 44294432 (1987); and Wagner et al., Proc. Natl. Acad. Sci. USA 87, 3410-3414 (1990). For review of gene marking and gene therapy protocols see Anderson et al., Science 256, 808-813 (1992).

In a preferred embodiment, the SLIM proteins, nucleic acids, modified proteins and cells containing the native or modified SLIM proteins are used in screening assays. Identification of this important modulator of lymphocyte activation permits the design of drug screening assays for compounds that modulate SLIM activity.

Screens may be designed to first find candidate agents that can bind to SLIM proteins, and then these agents may be used in assays that evaluate the ability of the candidate agent to modulate SLIM activity. Thus, as will be appreciated by those in the art, there are a number of different assays which may be run including a variety of binding assays and activity assays.

Thus, in a preferred embodiment, the methods comprise combining a SLIM protein and a candidate bioactive agent, and determining the binding of the candidate agent to the SLIM protein. Preferred embodiments utilize the human SLIM protein, although other mammalian proteins may also be used, including rodents (mice, rats, hamsters, guinea pigs, etc.), farm animals (cows, sheep, pigs, horses, etc.) and primates. These latter embodiments may be preferred in the development of animal models of human disease. In some embodiments, as outlined herein, variant or derivative SLIM proteins may be used, including deletion SLIM proteins as outlined above.

Furthermore, included within the definition of SLIM proteins are portions of SLIM proteins; that is, either the full-length protein may be used, or functional portions thereof. In addition, the assays described herein may utilize either isolated SLIM proteins or cells comprising the SLIM proteins, or cell free lysates comprising SLIM proteins.

Accordingly, in one aspect of the invention, methods for screening for bioactive agents capable of binding to SLIM are provided. In a preferred embodiment, the methods comprise combining SLIM and a candidate bioactive agent and determining the binding of candidate agent to SLIM. In one aspect, the method involves identifying the candidate agent.

Generally, in a preferred embodiment of the methods herein, the SLIM protein or the candidate agent is non-diffusably bound to an insoluble support having isolated sample receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the SLIM protein is bound to the support, and a candidate bioactive agent is added to the assay. Alternatively, the candidate agent is bound to the support and the SLIM protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The term “candidate bioactive agent” or “exogeneous compound” as used herein describes any molecule, e.g., protein, oligopeptide, small organic molecule, polysaccharide, polynucleotide, etc., with the capability of directly or indirectly altering the bioactivity of SLIM. Generally a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, preferably small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, a library of different candidate bioactive agents are used. Preferably, the library should provide a sufficiently structurally diverse population of randomized agents to effect a probabilistically sufficient range of diversity to allow binding to a particular target. Accordingly, an interaction library should be large enough so that at least one of its members will have a structure that gives it affinity for the target. Although it is difficult to gauge the required absolute size of an interaction library, nature provides a hint with the immune response: a diversity of 10⁷-10⁸ different antibodies provides at least one combination with sufficient affinity to interact with most potential antigens faced by an organism. Published in vitro selection techniques have also shown that a library size of 10⁷ to 10⁸ is sufficient to find structures with affinity for the target. A library of all combinations of a peptide 7 to 20 amino acids in length, such as generally proposed herein, has the potential to code for 20⁷ (10⁹) to 20²⁰. Thus, with libraries of 10⁷ to 10⁸ different molecules the present methods allow a “working” subset of a theoretically complete interaction library for 7 amino acids, and a subset of shapes for the 20²⁰ library. Thus, in a preferred embodiment, at least 10⁶, preferably at least 10⁷, more preferably at least 10⁸ and most preferably at least 10⁹ different sequences are simultaneously analyzed in the subject methods. Preferred methods maximize library size and diversity.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of prokaryotic and eukaryotic proteins may be made for screening against SLIM. Particularly preferred in this embodiment are libraries of bacterial, fungal, viral, and mammalian proteins, with the latter being preferred, and human proteins being especially preferred.

In a preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

In a preferred embodiment, the candidate bioactive agents are nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meieret al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Left. 4:395 (1994); Jeffs et al., J. Biomolecular NMR 34:17 (1994); Tetrahedron Left. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occuring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

As described above generally for proteins, nucleic acid candidate bioactive agents may be naturally occurring nucleic acids, random nucleic acids, or “biased” random nucleic acids. For example, digests of prokaryotic or eukaryotic genomes may be used as is outlined above for proteins.

In a preferred embodiment, the candidate bioactive agents are organic chemical moieties, a wide variety of which are described in the literature.

In a preferred embodiment, the candidate bioactive agents are linked to a fusion partner. By “fusion partner” or “functional group” herein is meant a sequence that is associated with the candidate bioactive agent, that confers upon all members of the library in that class a common function or ability. Fusion partners can be heterologous (i.e. not native to the host cell), or synthetic (not native to any cell. Suitable fusion partners include, but are not limited to: a) presentation structures, which provide the candidate bioactive agents in a conformationally restricted or stable form; b) targeting sequences, which allow the localization of the candidate bioactive agent into a subcellular or extracellular compartment; c) rescue sequences which allow the purification or isolation of either the candidate bioactive agents or the nucleic acids encoding them; d) stability sequences, which confer stability or protection from degradation to the candidate bioactive agent or the nucleic acid encoding it, for example resistance to proteolytic degradation; e) dimerization sequences, to allow for peptide dimerization; or f) any combination of a), b), c), d), and e), as well as linker sequences as needed.

In one embodiment of the methods described herein, portions of SLIM proteins are utilized. In a preferred embodiment, portions of SLIM having the ability to bind to CbI are used; such portions comprise the C-terminal domain of SLIM. In another preferred embodiment, portions of SLIM comprising an N-terminal SH2 domain are used; preferably such portions are able to bind to SLIM binding partners comprising phosphotyrosine. In another preferred embodiment, portions of SLIM comprising an N-terminal SH3 domain are used; preferably such portions are able to bind to SLIM binding partners comprising proline rich regions. In addition, the assays described herein may utilize either isolated SLIM proteins or cells comprising the SLIM proteins.

The determination of the binding of the candidate bioactive agent to the SLIM protein may be done in a number of ways. In a preferred embodiment, the candidate bioactive agent is labelled, and binding determined directly. For example, this may be done by attaching all or a portion of the SLIM protein to a solid support, adding a labelled candidate agent (for example a fluorescent label), washing off excess reagent, and determining whether the label is present on the solid support. Various blocking and washing steps may be utilized as is known in the art.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the proteins (or proteinaceous candidate agents) may be labeled at tyrosine positions using ¹²⁵I, or with fluorophores.

Alternatively, more than one component may be labeled with different labels; using ¹²⁵I for the proteins, for example, and a fluorophor for the candidate agents.

In a preferred embodiment, the binding of candidate bioactive agent is determined through the use of competitive binding assays. In this embodiment, the competitor is a binding moiety known to bind to the target molecule (i.e. SLIM), such as an antibody, peptide, binding partner, ligand, etc. In a preferred embodiment, the competitor is CbI. Under certain circumstances, there may be competitive binding as between the bioactive agent and the binding moiety, with the binding moiety displacing the bioactive agent. This assay can be used to determine candidate agents which interfere with binding between SLIM proteins and SLIM binding moieties, such as CbI. “Interference of binding” as used herein means that native binding of SLIM protein differs in the presence of the candidate agent. The binding can be eliminated or can be with a reduced affinity. Therefore, in one embodiment, interference is caused by, for example, a conformational change, rather than direct competition for the native binding site.

Accordingly, provided herein are methods for screening for a bioactive agent capable of modulating the binding of SLIM. In one embodiment, the methods comprise combining a candidate bioactive agent, SLIM protein, and a SLIM binding partner which will bind to SLIM in the absence of candidate agent and determining the binding of SLIM to binding partner in the presence of candidate bioactive agent. In a preferred aspect, the SLIM binding partner is CbI. The methods may additionally comprise determining the binding of SLIM to SLIM binding partner in the absence of candidate bioactive agent.

The methods may additionally comprise identifying the candidate agent. In one embodiment, SLIM and SLIM binding partner are combined first. Candidate agents may increase, decrease or otherwise alter the binding of SLIM to SLIM binding partner.

In one embodiment, the candidate bioactive agent is labeled. Either the candidate bioactive agent, or the competitor, or both, is added first to the protein for a time sufficient to allow binding, if present. Incubations may be performed at any temperature which facilitates optimal activity, typically between 4° C. and 40° C. Incubation periods are selected for optimum activity, but may also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate bioactive agent. Displacement of the competitor is an indication that the candidate bioactive agent is binding to the SLIM protein and thus is capable of binding to, and potentially modulating, the activity of the SLIM protein. In this embodiment, either component can be labeled. Thus, for example, if the competitor is labeled, the presence of label in the wash solution indicates displacement by the agent. Alternatively, if the candidate bioactive agent is labeled, the presence of the label on the support indicates displacement.

In an alternative embodiment, the candidate bioactive agent is added first, with incubation and washing, followed by the competitor. The absence of binding by the competitor may indicate that the bioactive agent is bound to the SLIM protein with a higher affinity. Thus, if the candidate bioactive agent is labeled, the presence of the label on the support, coupled with a lack of competitor binding, may indicate that the candidate agent is capable of binding to the SLIM protein.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Screening for agents that modulate the activity of SLIM may also be done. In a preferred embodiment, methods for screening for a bioactive agent capable of modulating the activity of SLIM comprise the steps of adding a candidate bioactive agent to a sample of SLIM, as above, and determining an alteration in the biological activity of SLIM. “Modulating the activity of SLIM” includes an increase in activity, a decrease in activity, or a change in the type or kind of activity present. Thus, in this embodiment, the candidate agent should both bind to SLIM (although this may not be necessary), and alter its biological or biochemical activity as defined herein. The methods include both in vitro screening methods, as are generally outlined above, and in vivo screening of cells for alterations in the presence, distribution, activity or amount of SLIM.

Thus, in this embodiment, the methods comprise combining a SLIM sample and a candidate bioactive agent, and evaluating the effect on SLIM activity. In a preferred embodiment, the activity of the SLIM protein is increased; in another preferred embodiment, the activity of the SLIM protein is decreased.

Thus, bioactive agents that are antagonists are preferred in some embodiments, and bioactive agents that are agonists may be preferred in other embodiments.

By “SLIM activity” or “SLIM bioactivity” or grammatical equivalents herein is meant at least one biological activity of SLIM protein and/or SLIM nucleic acid, including, but not limited to binding to CbI, binding to at least one phosphotyrosine containing SLIM binding partner (which binding partners include tyrosine kinases and phosphatases that associate with antigen receptor in lymphocytes), binding to at least one SLIM binding partner comprising a proline rich region (which binding partners include tyrosine kinases and phosphatases that associate with antigen receptor in lymphocytes); modulation of antigen receptor-induced lymphocyte activation; modulation of surface marker expression associated with lymphocyte activation, particularly CD69 expression; modulation of promoter activity associated with lymphocyte activation, particularly NFAT-responsive promoter activity; modulation of lymphocyte proliferation; and binding to tyrosine phosphorylated proteins following antigen receptor activation.

In one embodiment, SLIM proteins and nucleic acids that are able to modulate the basal activity of lymphocytes, i.e. modulation of activation in the absence of antigen-receptor induced lymphocyte activation or other induced lymphocyte activation, particularly in T-lymphocytes, are provided.

In one embodiment, SLIM proteins and nucleic acids that are able to modulate lymphocyte activation induced by ionomycin or ionomycin and PMA, particularly T lymphocyte activation, are provided.

In a preferred embodiment, SLIM proteins that are able to modulate antigen receptor-induced lymphocyte activation, but not able to modulate the expression of activation markers in response to ionomycin or ionomycin and PMA are provided.

Accordingly, in one aspect of the invention, methods for screening for bioactive agents capable of modulating the activity of SLIM are provided.

In a preferred embodiment, the methods comprise contacting a candidate bioactive agent to a cell comprising recombinant SLIM nucleic acid and expressing SLIM protein. In another preferred embodiment, the methods comprise contacting a library of candidate bioactive agents to a plurality of cells comprising recombinant SLIM nucleic acid and expressing SLIM protein.

A preferred method comprises expressing SLIM nucleic acid in a lymphocyte or mast cell, contacting the expressing cell with a candidate agent, and determining the level of expression of a surface marker which expression is normally correlated with lymphocyte or mast cell activation, in the presence of candidate agent. The methods may additionally comprise determining the level of surface marker expression in the absence of candidate agent. Preferred surface markers include CD23, CD69, CD80 and CD86. CD69 is especially preferred.

Another preferred method comprises expressing SLIM nucleic acid in a lymphocyte or mast cell, contacting the expressing cell to a candidate agent, and determining the level of activity of a promoter which activity normally correlates with lymphocyte or mast cell activation, in the presence of candidate agent. The methods may additionally comprise determining the level of promoter activity in the absence of candidate agent. Preferred promoters include NFAT-responsive promoters and the immunoglobulin heavy chain gene promoter. NFAT-responsive promoters are especially preferred.

In some embodiments, the methods involve determining lymphocyte activation. As will be appreciated, lymphocyte activation as well as non-lymphocyte leukocyte activation and platelet activation can be determined in a number of ways. It will be appreciated that mechanisms of leukocyte activation and methods for determining activation are known (see for example Kay, Immunol. Invest. 17:679-705, 1988; Lukacs et. al., Chem. Immunol. 72:102-120, 1999; Metcalf et. al., Physiol. Rev. 77:1033-1079, 1997; Hematol. Oncol. Clin. North Am. 4:1-26, 1990; Brass et. al., Adv. Exp. Med. Biol., 344:17-36, 1993; Brass et. al., Thromb. Haemost., 70:217-223, 1993; Cellular and Molecular Immunology, Abbas et. al., W. B. Saunders, ISBN 0-7216-3032-4, Chapters 7, 9, 12, and 14).

In one embodiment, indicators of lymphocyte activation are used. There are a number of parameters that may be evaluated or assayed to determine lymphocyte activation, including, but not limited to, immunoglobulin heavy chain gene promoter activity, NFAT activity, Ig secretion, IgG and IgM production, lymphocyte proliferation, expression cell surface markers correlated with lymphocyte activation, cytokine production, release of calcium from intracellular stores, amount of SYK protein, level of SYK protein ubiquitination, SYK protein tyrosine kinase activity, and ubiquitin specific protease activity directed to ubiquitin-conjugated SYK protein. These parameters may be assayed and used as indicators to evaluate the effect of candidate drug agents on lymphocyte activation. In this manner, rapid, accurate screening of candidate agents may be performed to identify agents that modulate lymphocyte activation.

In some embodiments, the assays include exposing lymphocytes comprising recombinant SLIM protein to a T-cell or B-cell activation agent that will induce T-cell or B-cell activation in the absence of candidate agent and recombinant SLIM protein. Alternatively, the cells may be exposed to conditions that normally result in T-cell and B-cell activation. The effect of the candidate agent on T-cell and B-cell activation is then evaluated. Preferred activation agents include anti-Igm antibody and anti-TCR antibody, preferably C308 monoclonal antibody (see Holland et. al., J. Ep. Med., 194:1263-1276, 2001, expressly incorporated herein by reference). These activation agents are sometimes referred to herein as “anti-Ig's”.

By a “population of cells” or “library of cells” herein is meant at least two cells, with at least about 10³ being preferred, at least about 10⁶ being particularly preferred, and at least about 10⁸ to 10⁹ being especially preferred. The population or sample can contain a mixture of different cell types from either primary or secondary cultures although samples containing only a single cell type are preferred, for example, the sample can be from a cell line, particularly tumor cell lines, as outlined below. The cells may be in any cell phase, either synchronously or not, including M, G1, S, and G2. In a preferred embodiment, cells that are replicating or proliferating are used; this may allow the use of retroviral vectors for the introduction of candidate bioactive agents. Alternatively, non-replicating cells may be used, and other vectors (such as adenovirus and lentivirus vectors) can be used. In addition, although not required, the cells are compatible with dyes and antibodies.

Preferred cell types for use in the invention include, but are not limited to, mammalian cells, including animal (rodents, including mice, rats, hamsters and gerbils), primates, and human cells, particularly including tumor cells of all types, including breast, skin, lung, cervix, colonrectal, leukemia, brain, etc. More preferable cell types include Jurkat cells and the Ig(+) and IgM secreting B cell lines CL-01, LA350 and CA46 as well as the human BJAB B cell line.

Preferred cell surface markers in the present invention exhibit low background expression in the absence of lymphocyte activation. Especially preferred cell surface markers include CD23, CD69, CD80, CD86.

Agents that recognize such surface molecules (e.g. antibodies) can be used as an affinity ligand, and attached to a solid support such as a bead,a surface,etc., and used to pull out cells that are undergoing T-cell and B-cell activation. Similarly, these agents can be coupled to a fluorescent dye such as PerCP, and then used as the basis of a fluorescence-activated cell sorting (FACS) separation.

FACS analysis can be used in conjunction with antibodies recognizing lymphocyte surface markers that are correlated with lymphocyte activation. FACS analysis is used to determine expression of these markers in unstimulated and stimulated lymphocytes which may additionally or alternatively be exposed to cytokines.

Immunoglobulin heavy chain gene promoter activity and NFAT activity can be measured using lymphocyte clones comprising an immunoglobulin heavy chain or NFAT responsive promoter operably fused to a reporter gene. For example, a surface Ig(+), IgM secreting B cell line such as the CL-01, CA46, or LA350 cell line is transfected with a construct comprising GFP/2a/TK fusion under the control of an immunoglobulin heavy chain promoter, Eμ and 3′α enhancer elements. Stable transfectants (referred to herein as immunoglobulin heavy chain reporter cell lines) are selected and maintained in gancyclovir. Preferred immunoglobulin heavy chain reporter cell lines for use in the present invention exhibit low background GFP expression and strong basal activity and/or inducible activity in the presence of positive control. Such cell lines can be generated with the use of retroviral constructs.

Immunoglobulin heavy chain reporter cell lines are transfected with SLIM nucleic acids which are expressed in the cell lines. A FACS machine may be used to determine reporter gene (GFP) expression in SLIM-transfected immunoglobulin heavy chain cell lines, comparing reporter gene expression in cells exposed to anti-Ig and not exposed to anti-Ig. In one embodiment, SLIM protein affects basal reporter gene expression. In a preferred embodiment, reporter gene expression is determined in the presence and absence of candidate bioactive agents in immunoglobulin heavy chain cell lines stimulated and not stimulated with anti-Ig. In another preferred embodiment, reporter gene expression is determined in the presence and absence of candidate bioactive agents as they are tested for their ability to modulate the effect of SLIM protein on basal reporter gene expression, i.e. absent BCR or TCR stimulation.

Release of intracellular calcium stores may be assayed using membrane permeant vital calcium sensing fluorescent dyes, as are well known in the art.

Some of the methods provided herein involve bypassing antigen receptor activation and activating intracellular calcium signaling, for example by treatment with ionomycin or ionomycin and the phorbol ester PMA, and assaying lymphocyte activation in the presence of candidate agent. Preferred SLIM proteins of the present invention include SLIM proteins that can modulate lymphocyte activation induced by calcium signals such as in response to ionomycin or ionomycin and PMA.

A preferred embodiment utilizes a cell proliferation assay. For example, B cells proliferate when activated. By “proliferation assay” herein is meant an assay that allows the determination that a cell population is either proliferating, i.e. replicating, or not replicating.

In a preferred embodiment, the proliferation assay is a dye inclusion assay. A dye inclusion assay relies on dilution effects to distinguish between cell phases. Briefly, a dye (generally a fluorescent dye as outlined below) is introduced to cells and taken up by the cells. Once taken up, the dye is trapped in the cell, and does not diffuse out. As the cell population divides, the dye is proportionally diluted. That is, after the introduction of the inclusion dye, the cells are allowed to incubate for some period of time; cells that lose fluorescence over time are dividing, and the cells that remain fluorescent are arrested in a non-growth phase.

The rate of loss of fluorescence is indicative of the rate of proliferation. An increase in proliferation rate above that of unstimulated cells is indicative of B cell activation.

Generally, the introduction of the inclusion dye may be done in one of two ways. Either the dye cannot passively enter the cells (e.g. it is charged), and the cells must be treated to take up the dye; for example through the use of a electric pulse. Alternatively, the dye can passively enter the cells, but once taken up, it is modified such that it cannot diffuse out of the cells. For example, enzymatic modification of the inclusion dye may render it charged, and thus unable to diffuse out of the cells. For example, the Molecular Probes CellTracker™ dyes are fluorescent chloromethyl derivatives that freely diffuse into cells, and then glutathione S-transferase-mediated reaction produces membrane impermeant dyes.

Suitable inclusion dyes include, but are not limited to, the Molecular Probes line of CellTracker™ dyes, including, but not limited to CellTracker™ Blue, CellTracker™ Yellow-Green, CellTracker™ Green, CellTracker™ Orange, PKH26 (Sigma), and others known in the art; see the Molecular Probes Handbook; chapter 15 in particular.

In general, inclusion dyes are provided to the cells at a concentration ranging from about 100 ng/ml to about 5 μg/ml, with from about 500 ng/ml to about 1 μg/ml being preferred. A wash step may or may not be used. In a preferred embodiment, a candidate bioactive agent is combined with the cells as described herein. The cells and the inclusion dye are incubated for some period of time, to allow cell division and thus dye dilution.

Also provided herein are methods for screening for an agent capable of modulating the ubiquitination of a CbI target protein that is capable of being ubiquitinated. In one aspect, the method comprises combining SLIM, a candidate bioactive agent, ubiquitin or polyubiquitin, CbI and a CbI target protein and determining the level of ubiquitination of target protein in the presence of candidate agent. The methods may additionally comprise determining the level of ubiquitination in the absence of candidate agent. In a preferred embodiment, CbI target protein is a SLIM binding partner. In a further preferred embodiment, CbI target protein is additionally a component of an antigen receptor in lymphocytes and/or mast cells. In another preferred embodiment, CbI target protein is a tyrosine kinase or phosphatase downstream of antigen receptor in lymphocytes and/or mast cells. The level of target protein ubiquitination may be determined by determining the amount of ubiquitin-conjugated target protein. Alternatively, the level of target protein ubiquitination may be determined by determining the amount of free ubiquitin.

The determination of ubiquitin-conjugated target protein may be done using standard protein preparation, immunoprecipitation, and Western blotting techniques in conjunction with standard ubiquitin determining techniques which include Western blotting. Such techniques are described herein. In addition, for example, a method for ubiquitinated protein determination is disclosed by Okada et al., Journal of Biological Chemistry 274:23787-23793, 1999, incorporated herein in its entirety by reference.

Alternatively, an in vitro assay using labeled ubiquitin and involving the determination of the amount of label associated with target protein may be done to determine the level of target protein ubiquitination in a screen for bioactive agents capable of altering the level of CbI target protein ubiquitination. Particularly preferred are high though put assays screening agents for their ability to alter the level of CbI target ubiquitination. In one embodiment, labeled ubiquitin or polyubiquitin is combined with a CbI protein as well as a ubiquitin activating enzyme, and a ubiquitin conjugating enzyme under conditions which provide for the ubiquitin loading of CbI (a ring finger protein and ubiquitin ligase). Alternatively ubiquitin conjugated CbI may be used. CbI target is added to the mixture. The amount of label associated with CbI target is determined, for example in a high throughput screen using a scintillation counter to determine the amount of radioactively labeled ubiquitin associated with CbI target protein in a sample. Alternatively, the amount of free labeled ubiquitin may be determined. The level of ubiquitin association with CbI target in the presence and absence of candidate agent is determined to identify an agent as being able to increase or decrease the level of target protein ubiquitination.

Alternatively, CbI target may be labeled and the amount of label associated with ubiquitin may be determined.

In vitro assays for determining the modulation of protein ubiquitination have been described. For example, U.S. Pat. No. 5,968,761 issued to Rolfe et al. sets forth in vitro methods for identifying inhibitors that prevent ubiquitination of a target protein. In addition, U.S. Pat. No. 5,976,849 issued to Hustad et al. provides similar methods.

In one embodiment, the CbI target used is SLIM.

In another embodiment, the methods comprise contacting a candidate agent with a cell comprising SLIM, CbI and CbI target protein, and determining the level of ubiquitination of CbI target protein in the presence and absence of candidate agent.

The proteins and nucleic acids provided herein can also be used for screening purposes wherein the protein-protein interactions of the SLIM proteins can be identified. Genetic systems have been described to detect protein-protein interactions. The first work was done in yeast systems, namely the “yeast two-hybrid” system. The basic system requires a protein-protein interaction in order to turn on transcription of a reporter gene. Subsequent work was done in mammalian cells. See Fields et al., Nature 340:245 (1989); Vasavada et al., PNAS USA 88:10686 (1991); Fearon et al., PNAS USA 89:7958 (1992); Dang et al., Mol. Cell. Biol. 11:954(1991); Chien et al., PNAS USA 88:9578(1991); and U.S. Pat. Nos. 5,283,173, 5,667,973, 5,468,614, 5,525,490, and 5,637,463. a preferred system is described in Ser. No. 09/050,863, filed Mar. 30, 1998 and Ser. No. 09/359,081 filed Jul. 22, 1999, entitled “Mammalian Protein Interaction Cloning System”. For use in conjunction with these systems, a particularly useful shuttle vector is described in Ser. No. 09/133,944, filed Aug. 14, 1998, entitled “Shuftle Vectors”.

In general, two nucleic acids are transformed into a cell, where one is a “bait” such as the gene encoding a SLIM protein or a portion thereof, and the other encodes a test candidate. Only if the two expression products bind to one another will an indicator, such as a fluorescent protein, be expressed. Expression of the indicator indicates when a test candidate binds to the SLIM protein, and identifies the candidate as being part of a T cell or B cell SLIM signaling pathway. A test candidate so identified may then be used as bait to identify binding proteins that are also identified as being part of a T cell or B cell SLIM signaling pathway. Additionally, SLIM proteins may be used to identify new baits, or agents thatbind to SLIM proteins. Additionally, the two-hybrid system can be used wherein a test candidate is added in addition to the bait and the SLIM protein encoding nucleic acids to determine agents which interfere with the binding of bait, such as SYK, to the SLIM protein.

In one embodiment, a mammalian two-hybrid system is preferred. Mammalian systems provide post-translational modifications of proteins which may contribute significantly to their ability to interact. In addition, a mammalian two-hybrid system can be used in a wide variety of mammalian cell types to mimic the regulation, induction, processing, etc. of specific proteins within a particular cell type. For example, proteins involved in a disease state (i.e., cancer, apoptosis related disorders) could be tested in the relevant disease cells. Similarly, for testing of random proteins, assaying them under the relevant cellular conditions will give the highest positive results. Furthermore, the mammalian cells can be tested under a variety of experimental conditions that may affect intracellular protein-protein interactions, such as in the presence of hormones, drugs, growth factors and cytokines, radiation, chemotherapeutics, cellular and chemical stimuli, etc., that may contribute to conditions which can effect protein-protein interactions, particularly those involved in cancer.

Assays involving binding such as the two-hybrid system may take into account non-specific binding proteins (NSB).

Expression in various cell types, and assays for SLIM activity are described above. The activity assays can be performed to confirm the activity of SLIM proteins which have already been identified by their sequence identity/similarity to SLIM, as well as to further confirm the activity of lead compounds identified as modulators of SLIM activity.

The components provided herein for the assays provided herein may also be combined to form kits. The kits can be based on the use of the protein and/or the nucleic acid encoding the SLIM proteins. In one embodiment, other components are provided in the kit. Such components include one or more of packaging, instructions, antibodies, and labels. Additional assays such as those used in diagnostics are further described below.

In this way, bioactive agents are identified. Compounds with pharmacological activity are able to enhance or interfere with the activity of the SLIM protein. The compounds having the desired pharmacological activity may be administered in a physiologically acceptable carrier to a host, as further described below.

The present discovery relating to the role of SLIM in leukocytes thus provides methods for inducing or preventing leukocyte activation, particularly lymphocyte activation. In a preferred embodiment, the SLIM proteins, and particularly SLIM protein fragments, are useful in the study or treatment of conditions which involve dysfunction or dysregulation of SLIM protein activity, i.e. to diagnose, treat or prevent SLIM associated disorders. Thus, “SLIM associated disorders” or “disease states” or “physiological states associated with SLIM dysfunction or dysregulation” include conditions involving insufficient, excessive, and inappropriate lymphocyte activation.

Thus, in one embodiment, methods for regulating lymphocyte activation in cells or organisms are provided. In one embodiment, the methods comprise administering to a cell or individual in need thereof, a SLIM protein in a therapeutic amount. Alternatively, an anti-SLIM antibody that reduces or eliminates the biological activity of the endogenous SLIM protein is administered. Alternatively and preferably a SLIM dominant negative protein variant is administered. In another preferred embodiment, a bioactive agent as identified by the methods provided herein is administered. In a further preferred embodiment, such an agent is a small molecule chemical composition which inhibits SLIM activity. Alternatively, the methods comprise administering to a cell or individual a recombinant nucleic add encoding an SLIM protein. In one embodiment, nucleic add encoding a SLIM dominant negative variant protein is administered. In another embodiment, a SLIM antisense nucleic acid is administered.

In one embodiment, the activity of SLIM is increased. As will be appreciated by those in the art, this may be accomplished in any number of ways. In a preferred embodiment, the activity of SLIM is increased by increasing the amount of SLIM in the cell, for example by overexpressing the endogenous SLIM or by administering a gene encoding a SLIM protein, using known gene-therapy techniques, for example. In a preferred embodiment, the gene therapy techniques include the incorporation of the exogenous gene using enhanced homologous recombination (EHR), for example as described in PCT/US93/03868, hereby incorporated by reference in its entirety.

In one embodiment, the activity of SLIM is decreased. As will be appreciated by those in the art, this may be accomplished in any number of ways. In a preferred embodiment, the activity of SLIM is decreased by decreasing the amount of SLIM mRNA in the cell, for example by expressing SLIM antisense RNA. Alternatively, endogenous SLIM activity is decreased by administering a dominant negative SLIM protein or a gene encoding a dominant negative SLIM protein. Alternatively, endogenous SLIM activity is decreased by administering anti-SLIM antibody or a gene encoding anti-SLIM antibody or an epitope recognizing portion thereof. Known gene-therapy techniques may be used to administer these agents. In a preferred embodiment, the gene therapy techniques involve incorporation of the exogenous gene into the host genome using enhanced homologous recombination (EHR), for example as described in PCT/US93/03868, hereby incorporated by reference in its entirety.

Without being bound by theory, it appears that SLIM protein is an important protein in leukocyte activation, particularly lymphocyte activation. Accordingly, disorders based on mutant or variant SLIM genes may be determined. In one embodiment, the invention provides methods for identifying cells containing variant SLIM genes comprising determining all or part of the sequence of at least one endogenous SLIM gene in a cell. As will be appreciated by those in the art, this may be done using any number of sequencing techniques. In a preferred embodiment, the invention provides methods of identifying the SLIM genotype of an individual comprising determining all or part of the sequence of at least one SLIM gene of the individual. This is generally done in at least one tissue of the individual, and may include the evaluation of a number of tissues or different samples of the same tissue. The method may include comparing the sequence of the sequenced SLIM gene to a known SLIM gene, i.e. a wild-type gene.

The sequence of all or part of the SLIM gene can then be compared to the sequence of a known SLIM gene to determine if any differences exist. This can be done using any number of known sequence identity programs, such as Bestfit, etc. In a preferred embodiment, the presence of a difference in the sequence between the SLIM gene of the patient and the known SLIM gene is indicative of a disease state or a propensity for a disease state.

In one embodiment, the invention provides methods for diagnosing a SLIM related condition in an individual. The methods comprise measuring the activity of SLIM in a tissue from the individual or patient, which may include a measurement of the amount or specific activity of a SLIM protein. This activity is compared to the activity of SLIM from either an unaffected second individual or from an unaffected tissue from the first individual. When these activities are different, the first individual may be at risk for a SLIM associated disorder. In this way, for example, monitoring of various disease conditions may be done, by monitoring the absolute SLIM activity in a sample or the specific activity of a SLIM protein from a sample. Similarly, activity levels may correlate with prognosis.

In one aspect, the expression levels of SLIM protein genes are determined in different patient samples or cells for which either diagnosis or prognosis information is desired. Gene expression monitoring is done on genes encoding SLIM proteins. In one aspect, the expression levels of SLIM protein genes are determined for different cellular states, such as normal cells and cells undergoing apoptosis or transformation. By comparing SLIM protein gene expression levels in cells in different states, information including both up- and down-regulation of SLIM protein genes is obtained, which can be used in a number of ways. For example, the evaluation of a particular treatment regime may be evaluated: does a chemotherapeutic drug act to improve the long-term prognosis in a particular patient. Similarly, diagnosis may be done or confirmed by comparing patient samples. Furthermore, these gene expression levels allow screening of drug candidates with an eye to mimicking or altering a particular expression level. This may be done by making biochips comprising sets of important SLIM protein genes, such as those of the present invention, which can then be used in these screens. These methods can also be done on the protein basis; that is, protein expression levels of the SLIM proteins can be evaluated for diagnostic purposes or to screen candidate agents. In addition, the SLIM protein nucleic acid sequences can be administered for gene therapy purposes, including the administration of antisense nucleic adds, or the SLIM proteins administered as therapeutic drugs.

SLIM protein sequences bound to biochips include both nucleic acid and amino acid sequences as defined above. In a preferred embodiment, nucleic acid probes to SLIM protein nucleic acids (both the nucleic acid sequences having the sequences outlined in the Figures and/or the complements thereof) are made. The nucleic acid probes attached to the biochip are designed to be substantially complementary to the SLIM protein nucleic acids, i.e. the target sequence (either the target sequence of the sample or to other probe sequences, for example in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein.

A “nucleic acid probe” is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. In some embodiments, much longer nucleic acids can be used, up to hundreds of bases (e.g., whole genes).

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can be covalent or non-covalent. By “non-covalent binding” and grammatical equivalents herein is meant one or more of either electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, etc. In general, the substrates allow optical detection and do not appreciably show fluorescence.

In a preferred embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference). In addition, in some cases, additional linkers, such as alkyl groups (including substituted and heteroalkyl groups) may be used.

In this embodiment, oligonucleotides, corresponding to the nucleic acid probe, are synthesized as is known in the art, and then attached to the surface of the solid support. As will be appreciated by those skilled in the art, either the 5′ or 3′ terminus may be attached to the solid support, or attachment may be via an internal nucleoside.

In an additional embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affimetrix GeneChip™ technology.

“Differential expression,” or grammatical equivalents as used herein, refers to both qualitative as well as quantitative differences in the genes' temporal and/or cellular expression patterns within and among the cells. Thus, a differentially expressed gene can qualitatively have its expression altered, including an activation or inactivation, in, for example, normal versus apoptotic cell. That is, genes may be turned on or turned off in a particular state, relative to another state. As is apparent to the skilled artisan, any comparison of two or more states can be made. Such a qualitatively regulated gene will exhibit an expression pattern within a state or cell type which is detectable by standard techniques in one such state or cell type, but is not detectable in both. Alternatively, the determination is quantitative in that expression is increased or decreased; that is, the expression of the gene is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology 14:1675-1680 (1996), hereby expressly incorporated by reference. Other techniques include, but are not limited to, quantitative reverse transcriptase PCR, Northern analysis and RNase protection.

As will be appreciated by those in the art, this may be done by evaluation at either the gene transcript, or the protein level; that is, the amount of gene expression may be monitored using nucleic acid probes to the DNA or RNA equivalent of the gene transcript, and the quantification of gene expression levels, or, alternatively, the final gene product itself (protein) can be monitored, for example through the use of antibodies to the SLIM protein and standard immunoassays (ELISAs, etc.) or other techniques, including mass spectroscopy assays, 2D gel electrophoresis assays, etc.

In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding an SLIM protein is detected by binding the digoxygenin with an anti-digoxygenin secondary antibody and developed with nitro blue tetrazolium and 5-bromo-4-chloro-3-indoyl phosphate.

In another preferred method, expression of SLIM protein is determined using in situ imaging techniques employing antibodies to SLIM proteins. In this method cells are contacted with from one to many antibodies to the SLIM protein(s). Following washing to remove non-specific antibody binding, the presence of the antibody or antibodies is detected. In one embodiment the antibody is detected by incubating with a secondary antibody that contains a detectable label. In another method the primary antibody to the SLIM protein(s) contains a detectable label. In another preferred embodiment each one of multiple primary antibodies contains a distinct and detectable label. This method finds particular use in simultaneous screening for a plurality of SLIM proteins. The label may be detected in a fluorometer which has the ability to detect and distinguish emissions of different wavelengths. In addition, a fluorescence activated cell sorter (FACS) can be used in this method. As will be appreciated by one of ordinary skill in the art, numerous other histological imaging techniques are useful in the invention and the antibodies can be used in ELISA, immunoblotting (Western blotting), immunoprecipitation, BIACORE technology, and the like.

In one embodiment, the SLIM proteins of the present invention may be used to generate polyclonal and monoclonal antibodies to SLIM proteins, which are useful as described herein. Similarly, the SLIM proteins can be coupled, using standard technology, to affinity chromatography columns. These columns may then be used to purify SLIM antibodies. In a preferred embodiment, the antibodies are generated to epitopes unique to the SLIM protein; that is, the antibodies show little or no cross-reactivity to other proteins. These antibodies find use in a number of applications. For example, the SLIM antibodies may be coupled to standard affinity chromatography columns and used to purify SLIM proteins as further described below. The antibodies may also be used as blocking polypeptides, as outlined above, since they will specifically bind to the SLIM protein.

The anti-SLIM protein antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the SLIM protein or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid a, synthetic trehalose dicorynomycolate). The immunization protocol may be selected by one skilled in the art without undue experimentation.

The anti-SLIM protein antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the SLIM protein or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against SLIM protein. Preferably, the binding specificity of monoclonal antibodies produced by the hybridoma cells is determined by immunoprecipitation or by an in vitro binding assay, such as radioimmunoassay (RIA) or enzyme-linked immunosorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein a-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

The anti-SLIM protein antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

Bispecific antibodies are monoclonal, preferably human or humanized, antibodies that have binding specificities for at least two different antigens. In the present case, one of the binding specificities is for the SLIM protein, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

The anti-SLIM protein antibodies of the invention have various utilities. For example, anti-SLIM protein antibodies may be used in diagnostic assays for an SLIM protein, e.g., detecting its expression in specific cells, tissues, or serum. Various diagnostic assay techniques known in the art may be used, such as competitive binding assays, direct or indirect sandwich assays and immunoprecipitation assays conducted in either heterogeneous or homogeneous phases [Zola, Monoclonal Antibodies: a Manual of Techniques, CRC Press, Inc. (1987) pp.147-158]. The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Anti-SLIM protein antibodies also are useful for the affinity purification of SLIM protein from recombinant cell culture or natural sources. In this process, the antibodies against SLIM protein are immobilized on a suitable support, such a Sephadex resin or filter paper, using methods well known in the art. The immobilized antibody then is contacted with a sample containing the SLIM protein to be purified, and thereafter the support is washed with a suitable solvent that will remove substantially all the material in the sample except the SLIM protein, which is bound to the immobilized antibody. Finally, the support is washed with another suitable solvent that will release the SLIM protein from the antibody.

The anti-SLIM protein antibodies may also be used in treatment. In one embodiment, the genes encoding the antibodies are provided, such that the antibodies bind to and modulate the SLIM protein within the cell.

In one embodiment, a therapeutically effective dose of an SLIM protein, agonist or antagonist is administered to a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for SLIM protein degradation, systemic versus localized delivery, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

SLIM proteins and nucleic acids are recognized herein as being critical regulators of B cell and T-cell activation.

Accordingly, in one aspect of the present invention, compositions useful for the treatment of disorders associated with lymphocyte activation are provided. Compositions include SLIM proteins and nucleic acids, as well as agents that bind to them and/or modulate their activity, including and preferably small molecule chemical compositions as discussed herein.

In one embodiment, compositions for treating disorders involving increased lymphocyte activation, where decreasing lymphocyte activation is desirable, are provided. Such disorders include chronic and acute inflammatory disease and autoimmune disease. Such compositions also find use as suppressants of normal immune responses where desired, for example as immunosuppressants in the treatment of response to transplantation. In a preferred embodiment, SLIM proteins comprising an amino acid sequence having at least about 90%, more preferably at least about 95%, more preferably at least about 98% identity to the amino acid sequence set forth in FIG. 2, and which will bind to CbI, are provided for use in treatment. In another embodiment, SLIM proteins comprising the amino acid sequence set forth in FIG. 2 are provided. Also provided are agonists of SLIM bioactivity, including small molecule chemical compounds.

In another embodiment, compositions for treating disorders involving decreased lymphocyte activation, where increasing lymphocyte activation is desirable, are provided. Such disorders include immunodeficiency disorders such as AIDS. In a preferred embodiment, SLIM variants are provided for use in treatment. Preferred variants include SLIM protein with a C-terminal deletion that is unable to bind CbI; SLIM protein lacking an N-terminal myristylation sequence which does not localize to the plasma membrane; SLIM protein that lacks an N-terminal SH2 domain and which is unable to bind SLIM binding partners comprising phosphotyrosine; and SLIM protein that lacks an N-terminal SH3 domain and which is unable to bind to SLIM binding partners comprising a proline rich motif. Antagonists of SLIM bioactivity, including small molecule chemical compounds, are also provided.

It is recognized that SLIM proteins and nucleic acids are modulators of lymphocyte proliferation, and that alterations in SLIM activity may produce alterations in proliferation, such as seen in cancer.

Accordingly, without being bound by theory, the present invention provides SLIM proteins and nucleic acids, as well as agents that bind to them and/or modulate their activity, including and preferably small molecule chemical compositions as discussed herein, which are useful in the treatment of T cell and B cell cancers.

It is recognized that signaling pathways involved in the regulation of cell proliferation frequently participate in, directly or indirectly, the regulation of cell survival and programmed cell death. It is further recognized that the dysregulation of mechanisms of programmed cell death can lead to cancer, particularly in lymphocytes. For example, overexpression of Bcl-2, which is involved in normal cell survival, is thought to be responsible for the survival of excessive numbers of lymphocytes in a form of lymphoma.

Without being bound by theory, the present invention provides SLIM proteins and nucleic acids, as well as agents binding to them or modulating their activity, including and preferably small molecule chemical compositions as discussed herein, which are useful in the treatment of disorders involving T cell and B cell survival and programmed cell death including cancer.

In a preferred embodiment, without being bound by theory, the present invention provides SLIM proteins and nucleic acids, as well as agents that bind to them and/or modulate their activity, including and preferably small molecule chemical compositions as discussed herein, which are useful in the treatment of physiological states that lead to the presentation of some or all symptoms characteristic of acute inflammatory disease, chronic inflammatory disease, autoimmune disease, or response to transplantation.

In a preferred embodiment, without being bound by theory, the present invention provides SLIM proteins and nucleic acids, as well as agents that bind to them and/or modulate their activity, including and preferably small molecule chemical compositions as discussed herein, which are useful as prophylactics directed to physiological states that lead to the presentation of some or all symptoms characteristic of acute inflammatory disease, chronic inflammatory disease, autoimmune disease, or response to transplantation.

The administration of the SLIM protein, agonist or antagonist of the present invention can be done in a variety of ways, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the composition may be directly applied as a solution or spray. Depending upon the manner of introduction, the compounds may be formulated in a variety of ways. The concentration of therapeutically active compound in the formulation may vary from about 0.1-100% wt.

The pharmaceutical compositions of the present invention comprise an SLIM protein, agonist or antagonist (including antibodies and bioactive agents as described herein, most preferably small molecule chemical compositions as described herein) in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

Combinations of the compositions may be administered. Moreover, the compositions may be administered in combination with other therapeutics, including growth factors or chemotherapeutics and/or radiation. Targeting agents (i.e. ligands for receptors on cancer cells) may also be combined with the compositions provided herein.

In one embodiment provided herein, the antibodies are used for immunotherapy, thus, methods of immunotherapy are provided. By “immunotherapy” is meant treatment of SLIM protein related disorders with an antibody raised against a SLIM protein. As used herein, immunotherapy can be passive or active. Passive immunotherapy, as defined herein, is the passive transfer of antibody to a recipient (patient). Active immunization is the induction of antibody and/or T-cell responses in a recipient (patient). Induction of an immune response can be the consequence of providing the recipient with an SLIM protein antigen to which antibodies are raised. As appreciated by one of ordinary skill in the art, the SLIM protein antigen may be provided by injecting an SLIM protein against which antibodies are desired to be raised into a recipient, or contacting the recipient with an SLIM protein nucleic acid, capable of expressing the SLIM protein antigen, under conditions for expression of the SLIM protein antigen.

In a preferred embodiment, a therapeutic compound is conjugated to an antibody, preferably an SLIM protein antibody. The therapeutic compound may be a cytotoxic agent. In this method, targeting the cytotoxic agent to apoptotic cells or tumor tissue or cells, results in a reduction in the number of afflicted cells, thereby reducing symptoms associated with apoptosis, cancer SLIM protein related disorders. Cytotoxic agents are numerous and varied and include, but are not limited to, cytotoxic drugs or toxins or active fragments of such toxins. Suitable toxins and their corresponding fragments include diptheria A chain, exotoxin A chain, ricin A chain, abrin A chain, curcin, crotin, phenomycin, enomycin and the like. Cytotoxic agents also include radiochemicals made by conjugating radioisotopes to antibodies raised against SLIM proteins, or binding of a radionuclide to a chelating agent that has been covalently attached to the antibody.

In a preferred embodiment, SLIM protein genes are administered as DNA vaccines, either single nucleic acids or combinations of SLIM protein genes. Naked DNA vaccines are generally known in the art; see Brower, Nature Biotechnology 16:1304-1305 (1998). Methods for the use of nucleic acids as DNA vaccines are well known to one of ordinary skill in the art, and include placing an SLIM protein gene or portion of an SLIM protein nucleic acid under the control of a promoter for expression in a patient. The SLIM protein gene used for DNA vaccines can encode full-length SLIM proteins, but more preferably encodes portions of the SLIM proteins including peptides derived from the SLIM protein. In a preferred embodiment a patient is immunized with a DNA vaccine comprising a plurality of nucleotide sequences derived from a SLIM protein gene. Similarly, it is possible to immunize a patient with a plurality of SLIM protein genes or portions thereof, as defined herein. Without being bound by theory, following expression of the polypeptide encoded by the DNA vaccine, cytotoxic T-cells, helper T-cells and antibodies are induced which recognize and destroy or eliminate cells expressing SLIM proteins.

In a preferred embodiment, the DNA vaccines include a gene encoding an adjuvant molecule with the DNA vaccine. Such adjuvant molecules include cytokines that increase the immunogenic response to the SLIM protein encoded by the DNA vaccine. Additional or alternative adjuvants are known to those of ordinary skill in the art and find use in the invention.

The examples described herein serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are expressly incorporated by reference in their entirety. Moreover, all sequences displayed, cited by reference or accession number in the references are incorporated by reference herein.

### EXAMPLES

Materials and Methods

Cell culture: Human BJAB B cells and Jurkat T cells (clone N) were routinely cultured in RPMI 1640 medium supplemented with 10% fetal calf serum (Hyclone), penicillin and streptamycin. Pheonix A cells in DMEM supplemented with 10% fetal calf serum, penicillin and streptamycin. To produce the tTA-BJAB cell line, BJAB cells were infected with retroviral construct which constitutively expresses the tetracycline transactivator protein and a reporter construct which expresses Ly2 driven by a tetracycline responsive element (TRE). The tTA-BJAB cell population was optimized by sorting multiple sounds for high TRE-dependent expression of Ly2 in the absence of Dox and strong repression of Ly2 expression in the presence Dox. The cells were also independently for maximal anti-Igm-induced expression of CD69. Doxycycline was used at a final concentration of long/ml for at least 6 days to downregulate expression of cDNAs from the TRE promoter.

Transfection and infection: Pheonix A packaging cells were transfected with retroviral vectors using calcium phosphate for 6 hours as standard protocols. After 24 hours, supernatant was replaced with complete RPMI medium and virus was allowed to accumulate for an additional 24 hours. Viral supernatant was collected, filtered through a 0.2 mM filter and mixed with BJAB cells at a density of 2.5×10⁵ cells/ml. Cells were spun at room temperature for 3 hours at 3000 rpm, followed by overnight incubation at 37° C. Transfection and infection efficiencies were monitored and functional analysis was carried out at least 4 days after infection. For transient transfection in Jurkat-N cells, 20-40 μg of indicated plasmids, plus 10 μg of NFAT-Luciferase in the case of the report assay, were electroporated into 10⁷ Jurkat cells, as previously described (Wu et. al., Mol. Cell. Biol., 15:4337-4346, 1995). Cells were cultured for 40 hrs after transfection and assayed as indicated.

Constructs and libraries: Dominant negative ΔSyk (amino acids 1-402), SLAP and SLIM were cloned into the retroviral pTRA-IRES.GFP vector (Leo et. al., Nat. Genet. 27:23-29, 2001; Lorens et. al., Mol. Ther. 1:438-447, 2000). For generating the myristylation mutant (SLIM-myr), the second a.a. residue G was substituted with A using PCR mutagenesis. The C-terminal truncation mutant (SLIM-ΔC) containing a.a. 1 to 194 was generated by standard PCR procedure. All three versions of SLIM were C-terminal tagged with a FLAG-epitope (DYKDDDDK) and cloned into both the retroviral pTRA-IRES.GFP vector and a mammalian expression vector pEFBOS-IRES.GFP. NFAT-luciferase was purchased from Clontech.

RNA extracted from lymph node, thymus, spleen and bone marrow was used to produce two cDNA libraries; one random primed and directionally cloned and the second non-directionally cloned and provided with 3 exogenous ATG in 3 frames. cDNAs were cloned into the pTRA-exs vector giving robust Doxycycline-regulable transcription of cDNAs from the TRE promoter. The total combined library complexity was 5×10⁷ independent clones.

Stimulation: For CD69 upregulation experiment, tTA-BJAB cells were split to 2.5×10⁵ cells/ml 24 hours prior to stimulation. Cells were spun and resuspended at 5×10⁵ cells/ml in fresh complete RPMI medium in the presence of 0.3 mg/ml anti-Igm F(ab′)₂ (Jackson Immunoresearch) for 20-26 hours at 37° C., and then assayed for surface CD69 expression. Jurkat-N T cells were stimulated with Leu4 (anti-CD3) at 1 μg/ml for 24 hr, and then assayed for CD69 expression. For tyrosine phosphorylation and immunoprecipitation, stably infected, GFP-sorted BJAB cells were stimulated with the anti-Igm F(ab′)₂ for 2 minutes at 37° C. in PBS. Cells were then lysed and analyzed.

Cell surface marker analysis: tTA-BJAB or Jurkat-N cells were stained with either an APC-conjugated or a PE-conjugated mouse monoclonal anti-human CD69 antibody (Caltag) at 4° C. for 20 minutes and analyzed using a Facscalibur instrument (Becton Dickinson) with Celiquest software. Cell sorts were performed on a MoFlo (Cytomation).

cDNA screen: Pheonix A packaging cells were transfected with a mixture of the two tTA regulated retroviral pTRA-exs cDNA libraries spiked with tTA-regulated pTRA-IRES.GFP-DSyk as an internal positive control. Supernatant containing packaged viral particles was used to infect tTA-BJAB cells with an efficiency of ˜80%. After 4 days of cDNA expression, library infected cells were stimulated with 0.3 mg/ml anti-Igm F(ab′)₂ for 20-26 hours, stained with APC-conjugated anti-CD69, and lowest CD69-expressing cells were isolated using a fluorescence activated cell sorter. Sorting was repeated multiple rounds allowing the cells to rest for 6 days between stimulations until the population was significantly enriched for non-responders. Single cells were deposited from three separate rounds of sorting. Cell clones were expanded in the presence and absence of Dox, stimulated and analyzed for CD69 upregulation.

Immunoprecipitation and immunoblots: Stimulated BJAB or Jurkat-N cells were lysed in NP-40 lysis buffer (1% NP40, 150 mM NaCl, 10 mM Tris-HCl pH 7.5, protease inhibitors and phosphatase inhibitors, as previously described. After 20 min at 4° C., lysates were centrifuged for 20 min at 14000 rpm and the supernatants were subjected to immunoprecipitation with the indicated antibodies. Resulting immune complexes were washed extensively in lysis buffer, resolved by SDS-PAGE and transferred to PVDF membranes. Blots were blocked with 5% albumin in TBST (10 mM Tris, pH 7.8, 150 mM NaCl, with 0.05% Tween detergent). Blots were incubated with the indicated antibodies following by a secondary antibody conjugated with horseradish peroxidase and then assayed by enhanced chemiluminescence assay (ECL kit, Amersham. Blots were stripped using 0.1M glycine, pH 2.5, washed extensively in TBST buffer, and then blocked and probed as described above. 4G10 monoclonal anti-phosphotyrosine antibodies were from UBI and anti-CbI was purchased from Santa Cruz Biotechnology.

Luciferase Assays: Transfected Jurkat-N cells (1×10⁵) were aliquoted into a 96-well plate (Corning) 40 hrs after transfection and cultured in a final volume of 100 μl RPMI growth medium. Cells were unstimulated or stimulated at 37° C. in the growth medium containing either 1:1000 dilution of C305 ascites or 50 ng/ml phorbol myristate acetate (PMA) and 1 μM ionomycin. After 12 hr. stimulation, cells were lysed in 5× lysis buffer (Promega) and assayed using the luciferase kit purchase from Promega. Luciferase activity was read on the Luminometer (Dynex Technology) and determined in triplicates for each experimental condition.

### EXAMPLE 1

### Genetic Screen for Regulators of Antigen Receptor Signaling in B Cells

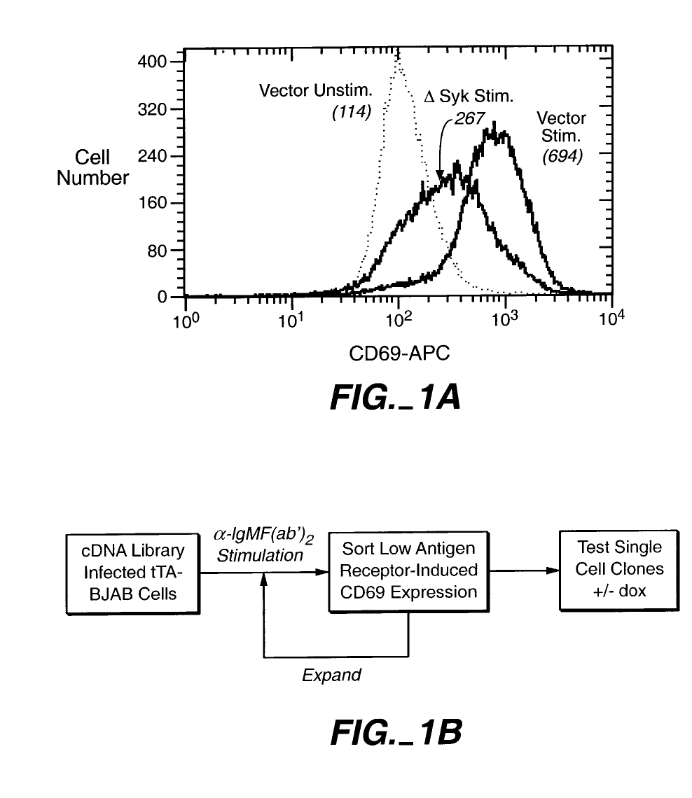

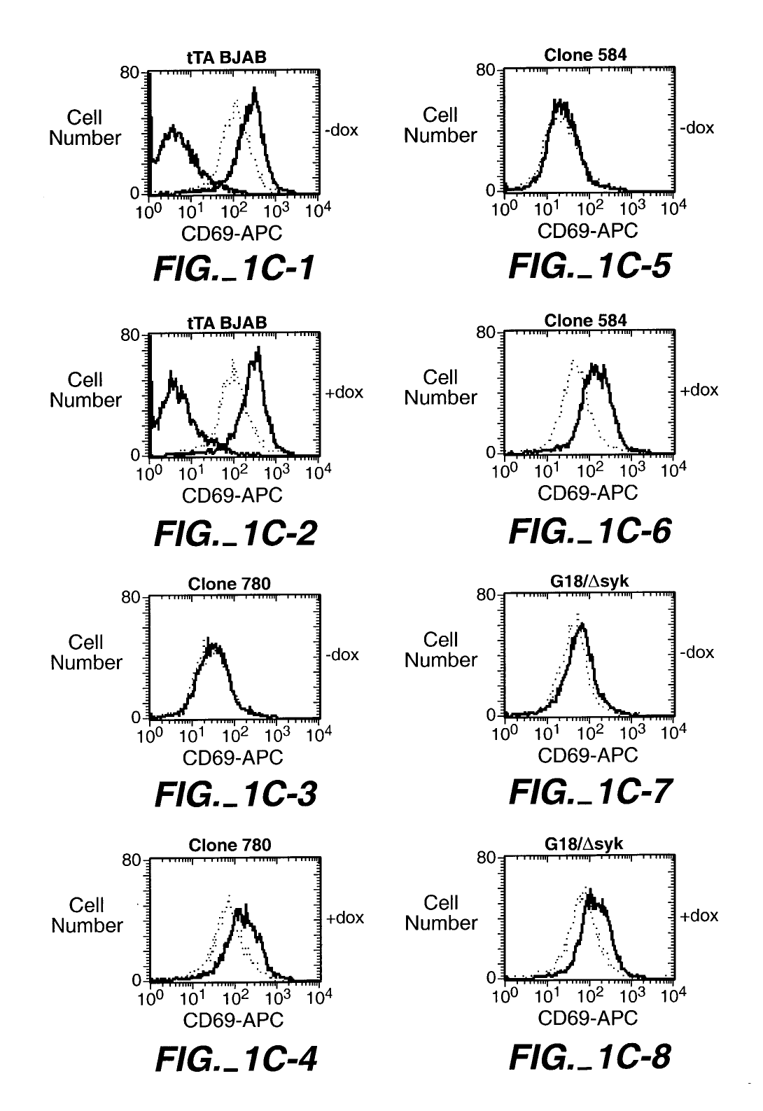

Two TRE-dependent retroviral cDNA expression libraries were constructed using mixed mRNAs extracted from lymph node, thymus, spleen and bone marrow tissues. The libraries were pooled, spiked with a tTA-regulated DSyk construct as an internal positive control to monitor enrichment of genetic inhibitors, and introduced into tTA-BJAB cells. Cells exhibiting the lowest anti-Igm-induced levels of CD69 expression were enriched by multiple rounds of fluorescence-based sorting and single cell cloned (FIG. 1B).

A total of 1394 single cell clones were expanded, replica plated and grown in the presence or absence of dox to allow the down regulation of TRE-dependent library cDNAs. Duplicates were stimulated with anti-Igm F(ab′)₂ and assayed for surface CD69 expression. Although many clones isolated from the sorts failed to exhibit reproducible inhibition after single cell cloning, we obtained over 120 clones that consistently exhibited a 50% inhibition of anti-Igm F(ab′)₂-induced CD69 expression in the absence (cDNA on) compared to the presence (cDNA off) of dox. When cDNAs were turned on, variable inhibition of CD69 upregulation was observed in different clones ranging from 50% to 90%.

The phenotypes of three representative positive clones (G18, 584, and 780), are illustrated in FIG. 1C. Clone G18 represents recovery of the dominant negative ΔSyk that was spiked into the library. cDNA sequence analysis of library inserts from additional positive clones identified a number of known regulators of antigen receptor signaling, including the inhibitor dual specificity phosphatase I, and deletional dominant negative versions of positive regulators such as the tyrosine kinase HCK, phospholipase Cg-1 and Syk (Table 1). The isolation of both the spiked ΔSyk and known antigen receptor regulators confirmed that our screening strategy indeed led to significant enrichment of potent inhibitors of BCR signaling.

### EXAMPLE 2

### Isolation of SLAP and a Novel SLAP Homologue, SLIM (SLAP-2)

Sequencing of retroviral library cDNA inserts recovered from clone 780 identified the Src-like adaptor protein, SLAP. SLAP has previously been described as a negative regulator of T cell receptor (TCR) signaling (Sosinowski et al) and the isolation of this cDNA not only validated the efficacy of the functional selection used, but also established its role in the BCR signaling pathway. Interestingly, clone 584 contained a cDNA encoding a novel protein of 261 amino acids (a.a.) (FIG. 2A). Sequence analysis revealed that it shares significant homology with SLAP, comprising an N-terminal myristylation consensus sequence, one SH2- and one SH3 domain, and a unique C-terminus but lacking an enzymatic domain (FIG. 2B). We therefore named it SLIM for Src-like inhibitory molecule. Subsequent analyses of an additional 140 clones identified another 7 SLIM-containing clones and 2 SLAP clones. All of the SLIM and SLAP inserts represented the entire open reading frame and were transcribed in the sense orientation, suggesting that SLIM is an inhibitory adapter.

Northern analysis of multiple tissues indicated that SLIM was predominantly expressed in peripheral blood leukocytes, spleen, thymus and lung. Abundant SLIM transcript was also detected in Molt 4 human T cell line, but not other tumor cell lines (FIG. 2C). RT-PCR revealed that SLIM was present in unstimulated and stimulated purified human T cells, B cells, and monocytes, but was absent in fibroblasts and epithelial cells (FIG. 2D, and data not shown). Taken together, SLIM appears to be a hematopoetic-specific signaling adapter.

### EXAMPLE 3

### SLIM Inhibits Antigen Receptor Signaling in B and T Lymphocytes

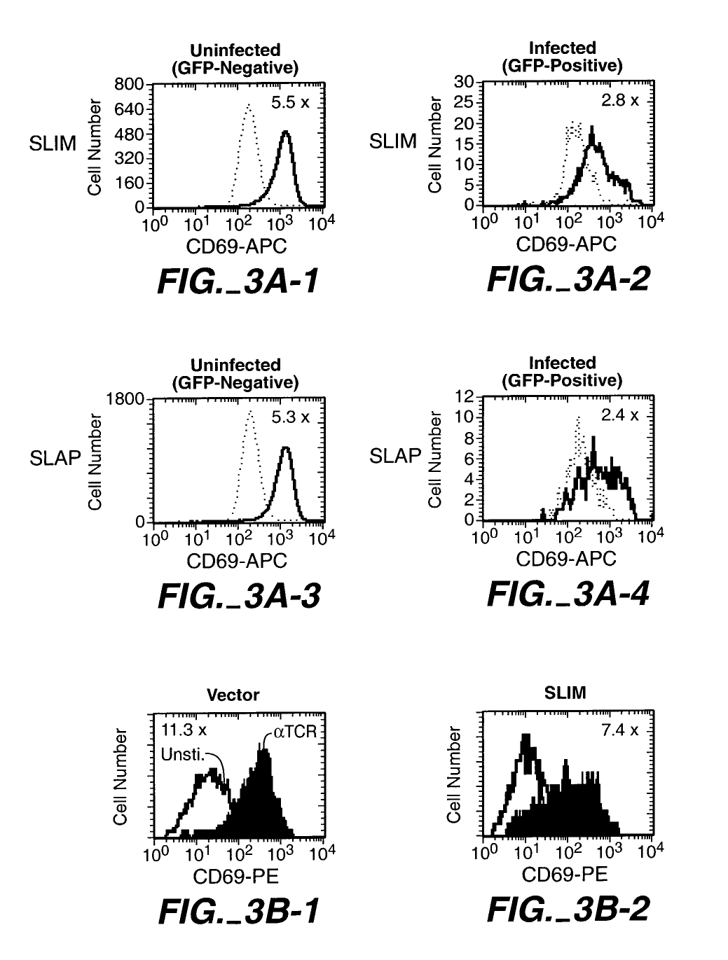

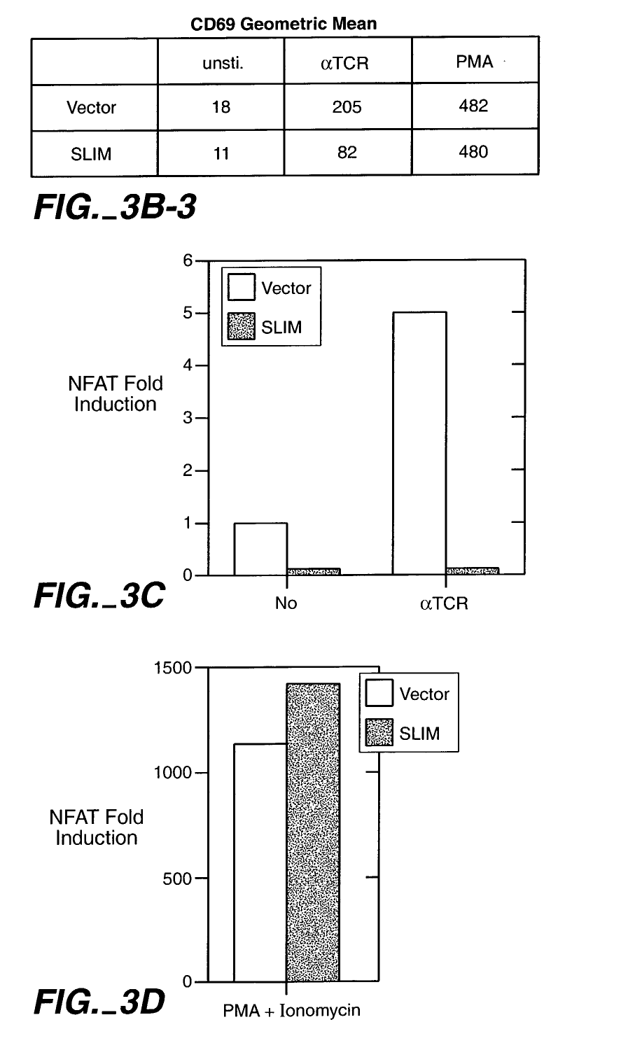

To confirm that the inhibitory phenotype observed in clones 584 and 780 was indeed contributed by the cDNA inserts, full length SLIM and SLAP cDNAs were subcloned into the pTRA-IRES.GFP retroviral vector (See Material and Methods) and reintroduced into parental tTA-BJAB cells (FIG. 3A). Co-expression of SLIM or SLAP with GFP allowed for parallel analysis of uninfected cells and infected the cells within the same sample, thus providing an internal control. Uninfected GFP-negative cells exhibited between a 5.3 and 5.5 fold up-regulation of anti-Igm-induced CD69 expression. In GFP-positive cells expressing either SLIM or SLAP, CD69 induction was reduced to 2.8 and 2.4 fold respectively, representing approximately half of the control values.

Since SLAP has previously been shown to inhibit signaling downstream of the TCR, the presence of SLIM in T cells led us to investigate whether SLIM is also capable of attenuating signals initiated from the TCR. SLIM was subcloned into a mammalian expression vector containing IRES.GFP (see Materials and Methods), which allowed us to monitor SLIM-expressing Jurkat T cells following transient transfection. As shown in FIG. 3B, TCR stimulation resulted in a marked 11.3 fold increase in CD69 surface expression in the control vector transfected Jurkat cells. However, expression of SLIM led to a significant 34% reduction in this response. The inhibitory effect induced by the overexpression of SLIM appeared to be specific to the TCR-stimulated pathway, since it did not affect the phorbol ester-induced CD69 induction (FIG. 3B). This observation implies that SLIM may function in the early signaling events following TCR stimulation. Taken together, these data demonstrate that, similar to SLAP, SLIM functions as a negative regulator of antigen receptor-mediated activation events including CD69 upregulation in both T and B cells.

Antigen receptor activation contributes to the production of many cytokines, including IL-2. Cis-acting elements in the IL-2 promoter bind proteins such as nuclear factor of activated T cells (NFAT). Reporter constructs containing multimers of the NFAT element are responsive to antigen receptor-mediated signaling and require activation of both the calcium and the MAP kinase pathways. To determine whether SLIM plays a role in TCR-triggered NFAT activation, we transiently overexpressed SLIM in Jurkat T cells and examined its effect on the NFAT-Luciferase reporter. As shown in FIG. 3C, overexpression of SLIM resulted in an almost complete inhibition of TCR-induced NFAT activation. In contrast, it did not block the NFAT activation induced by treatment with phorbol ester and ionomycin, two pharmacological agents that bypass early antigen receptor signaling. In summary, SLIM appears to play an important receptor proximal role in downregulating T and B cell-mediated responses.

Inhibition of CD69 upregulation by SLIM and SLAP in tTA-BJAB cells. Epitope-tagged SLIM and SLAP cDNAs in the pTRA-IRES.GFP vector were infected into naive tTA-BJAB cells. Surface CD69 expression was analyzed in unstimulated (dotted line) and anti-Igm F(ab′)₂-stimulated (solid line) cells. The ratio of the induced and uninduced geometric mean of APC-CD69 fluorescence is shown for uninfected (GFP negative) and infected (GFP positive) cells. Both SLIM and SLAP decrease anti-Igm F(ab′)₂-induced CD69 upregulation to approximately 50% of control values. (FIG. 3)

### EXAMPLE 4 (see FIG. 3)

### Anti-CD3 induced CD69 Expression is Inhibited by SLIM

Jurkat-N cells were transiently transfected with SLIM in pEFBOS-IRES.GFP or vector alone. Cells were stimulated with anti-CD3 Leu4 (1 μg/ml) or PMA (50 ng/ml) for 24 hrs and stained for CD69-PE. CD69 expression in GFP positive cells is represented graphically (unstimulated =line; stimulated solid) and the geometric mean values (fluorescence intensity) are summarized in the table.

Anti-CD3-induced NFAT promoter activation is inhibited by SLIM Jurkat-N cells were transiently co-transfected with SLIM in pEFBOS-IRES.GFP or vector alone, and an NFAT-Luciferase reporter construct. Cells were stimulated with anti-CD3, or PMA plus ionomycin for 12 hrs, and assayed for luciferase activity. Fold induction of luciferase activity over the unstimulated, vector-transfected sample is shown. The basal luciferase activity for this experiment was approximately 100 arbitrary light units (AU). The data are representative of two independent experiments.

### EXAMPLE 4

### The N-Terminal Myristylation Site and C-Terminal Unique Regions of SLIM are Required for Inhibition of Antigen Receptor Signaling

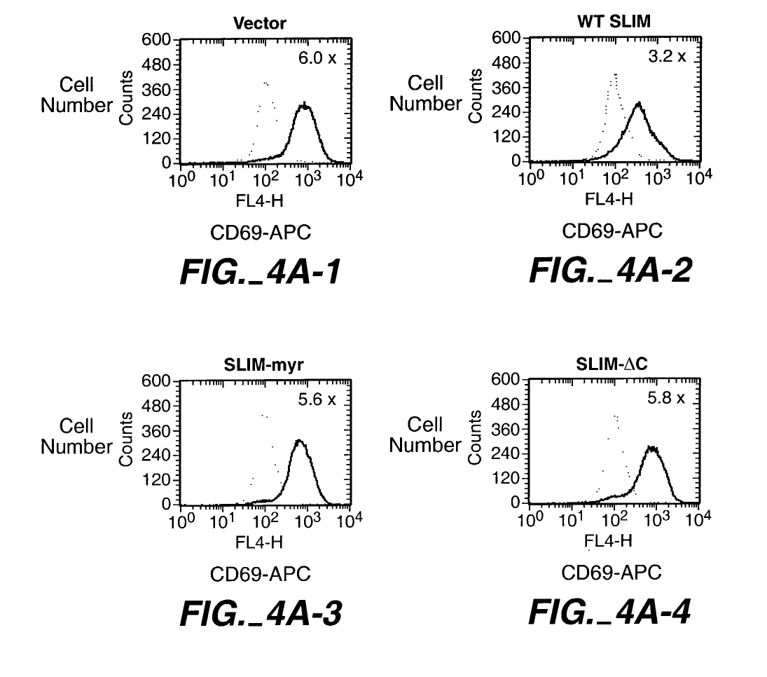

The proximal signaling role of SLIM suggests it may require localization to the membrane compartment in order to interact with components of the antigen receptor signaling complex. Myristylation has been shown to localize signaling proteins including Src family kinases to the plasma membrane and in many cases to be critical for their function. Since SLIM contains an N-terminal consensus motif (MG₁₋₄T/S) for addition of a myristyl group, we generated an amino acid substitution in the myristylation motif within a FLAG-tagged full length SLIM (SLIM-myr) and examined its effect on antigen receptor-induced signaling. SLIM-myr in the pTRA-IRES.GFP vector was introduced into tTA-BJAB cells. The infected GFP-positive cells were sorted and assayed for anti-Igm induced CD69 expression (FIG. 4A). In contrast to cells expressing wild type SLIM, CD69 induction in cells expressing SLIM-myr was comparable to the vector infected control, demonstrating that the myristylation sequence, and most likely its membrane association, plays a critical role in SLIM function.

The C terminus of SLAP has been proposed to perform a number of important functions including mediating dimerization, interacting with signaling partners (Tang et al), and determining solubility in mild detergents (Sosinowski et al). As this region of SLIM exhibits the highest degree of sequence divergence from SLAP, we expressed a truncated form of SLIM (SLIM-ΔC) in tTA-BJAB cells and examined its effect on antigen-induced CD69 upregulation. Similar to those expressing SLIM-myr, SLIM-ΔC-expressing tTA-BJAB cells exhibited approximately normal induction of CD69 in response to anti-Igm stimulation (FIG. 4A), although some inhibition was observed when the data was gated on the highest GFP-expressing cells (data not shown). This result suggests that although the C-terminus is important for SLIM function, its loss can be partially compensated by higher level of protein overexpression.

Epitope-tagged wild type SLIM, SLIM-myr and SLIM-DC in the pTRA-IRES.GFP vector were stably introduced into tTA-BJAB cells, and GFP-positive cells were purified by sorting. Cells were stimulated with anti-Igm F(ab′)₂ and stained for surface expression of CD69. Wild type SLIM decreased CD69 induction to 53% of the control value, whereas SLIM-myr and SLIM-ΔC resembled the vector control. The ratio of the induced and uninduced geometric means is shown. (FIG. 4)

### EXAMPLE 5

### SLIM Associates with Tyrosine Phosphorylated Proteins Including CbI After Antigen Stimulation

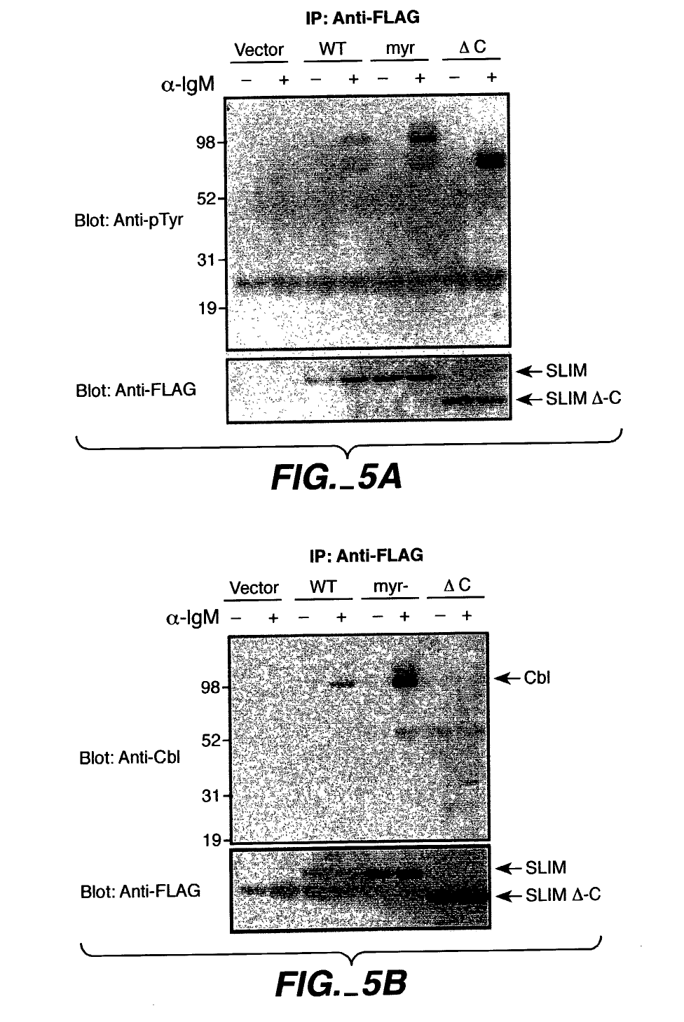

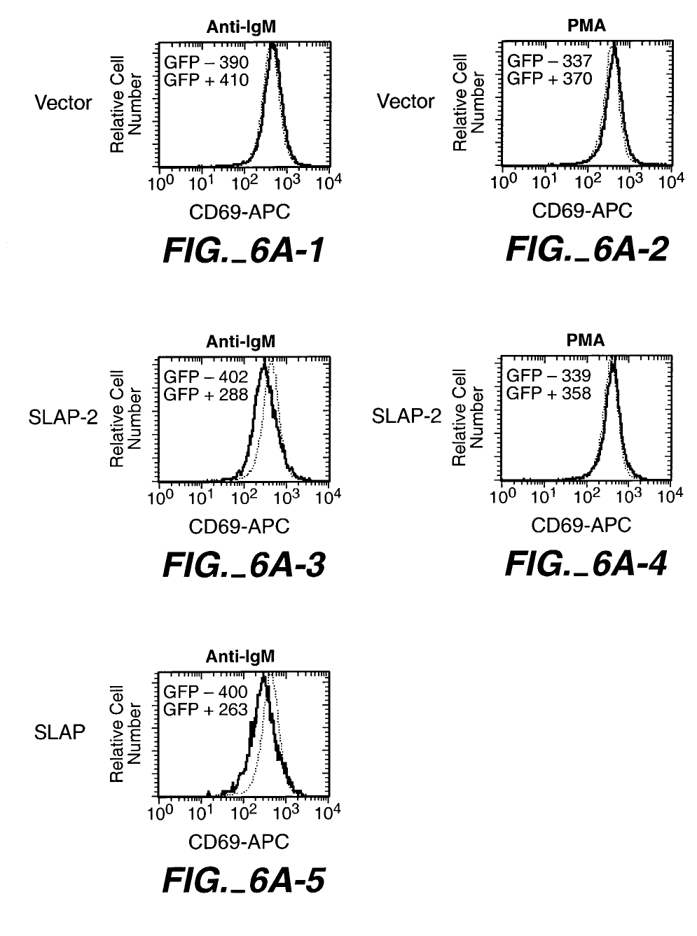

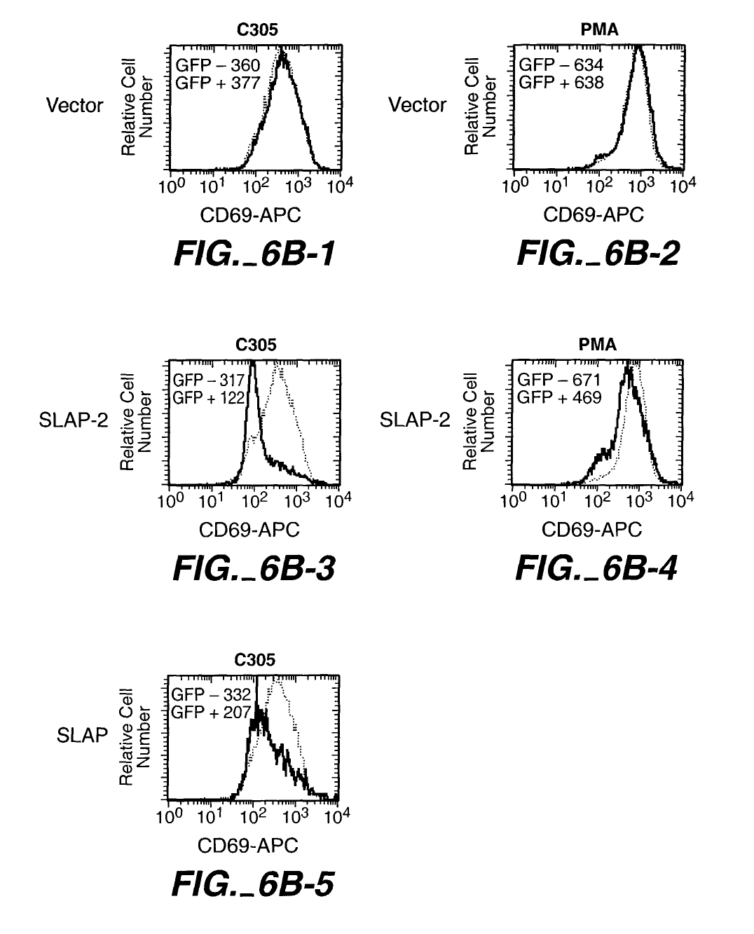

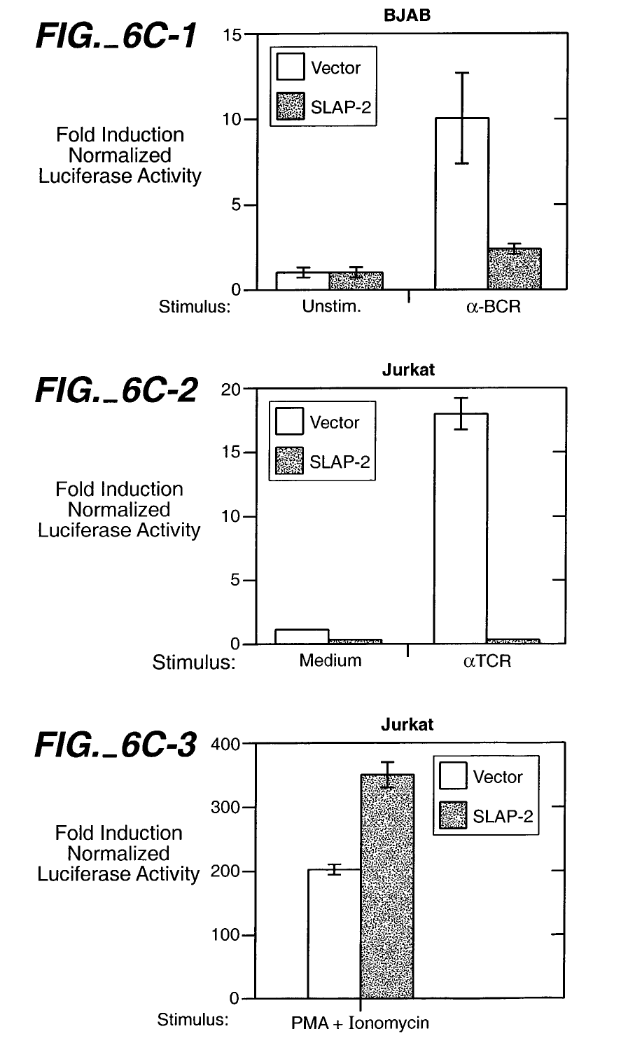

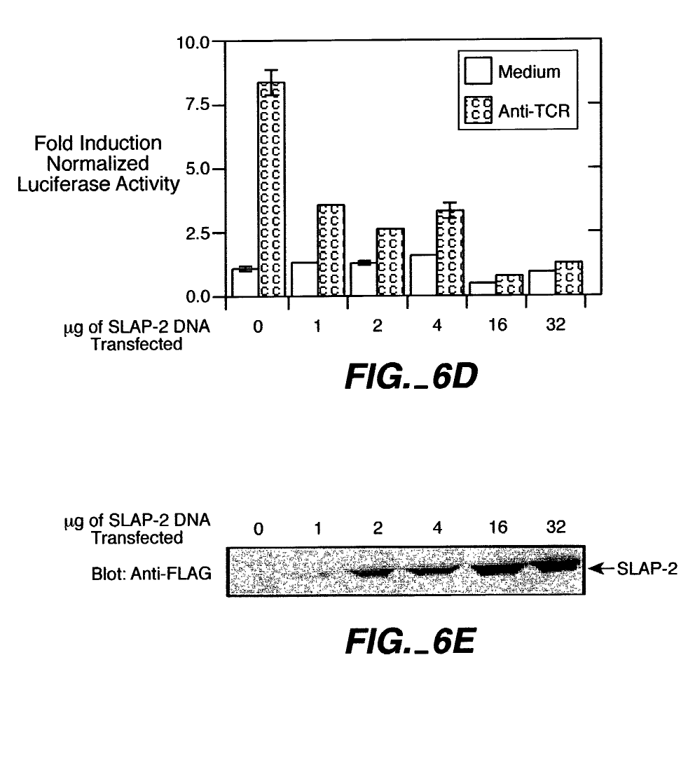

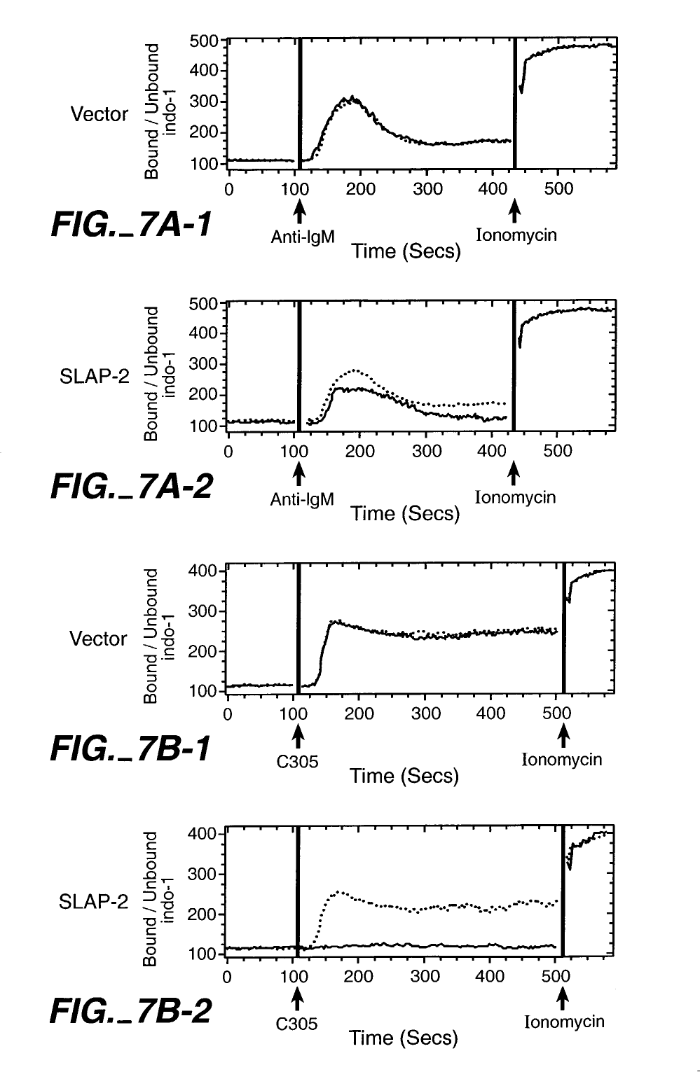

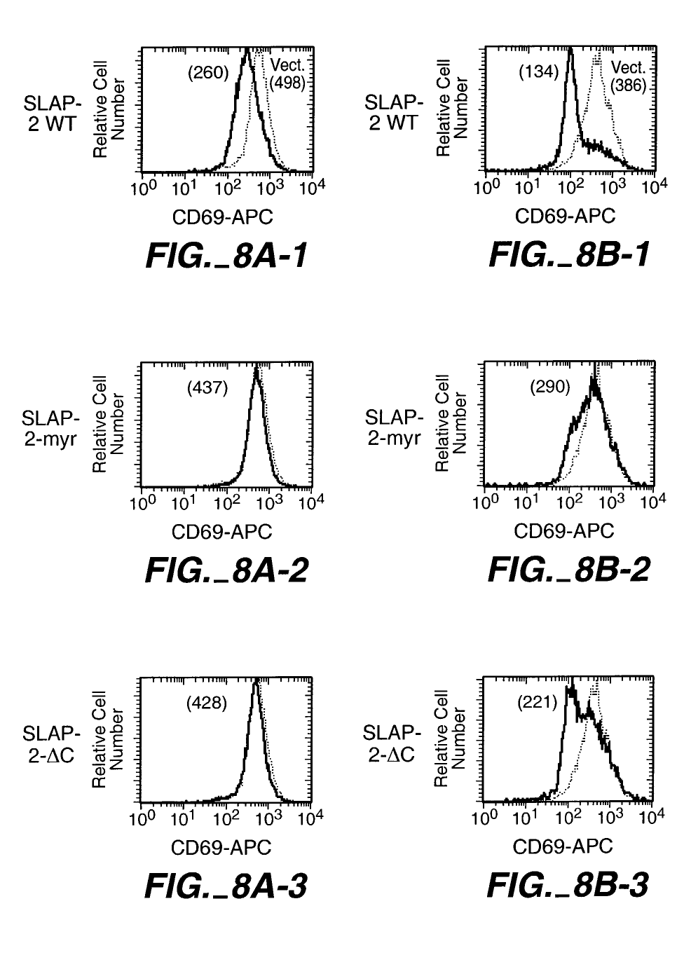

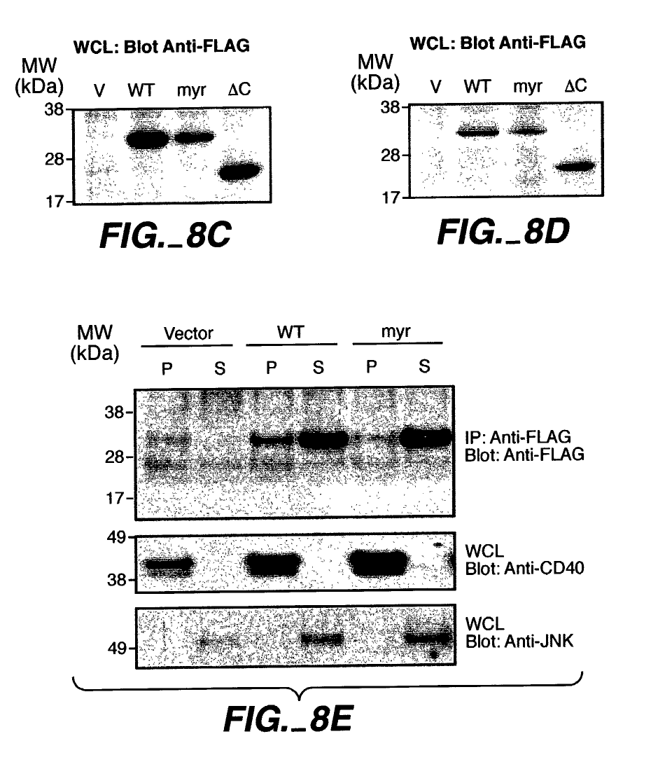

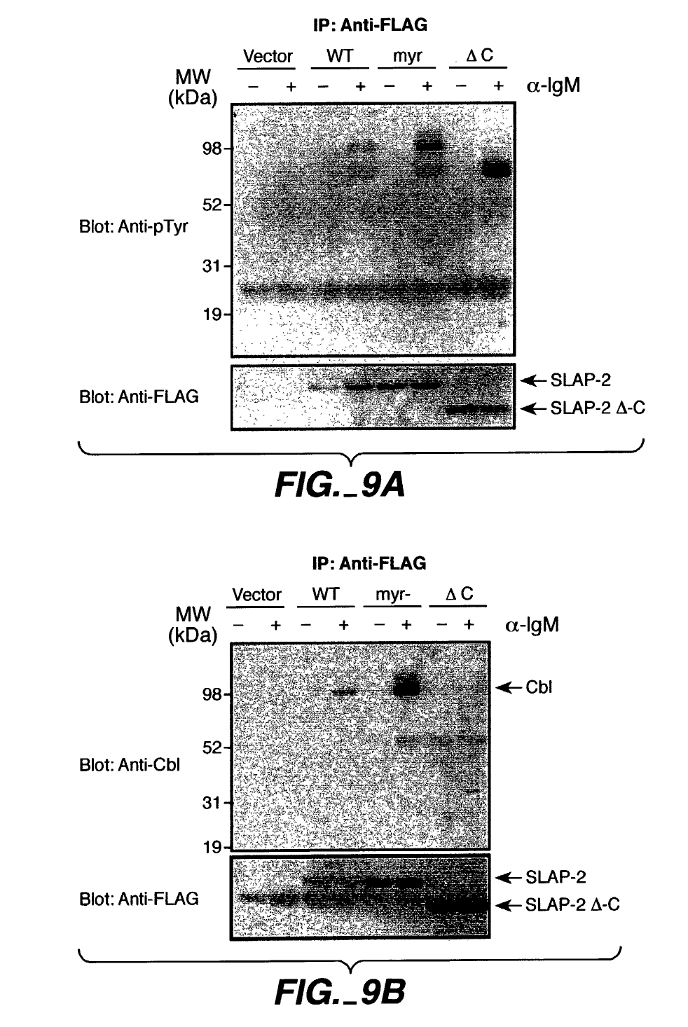

SLIM associates with tyrosine phosphorylated proteins following antigen receptor engagement. Signaling through the TCR or BCR results in a rapid increase in tyrosine phosphorylation of numerous intracellular proteins, initiated by Src family and Syk/ZAP70 kinase activation. These early signaling events ultimately result in transcriptional activation, upregulation of surface antigens, and other lymphocyte effector functions. Adapter proteins play an important intermediary role in integrating upstream signals to produce biological function. In order to investigate the nature of SLIM signaling complexes, epitope-tagged versions of SLIM, SLIM-myr, or SLIM-DC were stably introduced into BJAB cells. All three proteins became associated with a number of tyrosine phosphorylated proteins following BCR stimulation (FIG. 5A), demonstrating that SLIM indeed participates in BCR signaling pathways. Interestingly, a prominent phosphoprotein of approximately 110 kD was absent in the immunoprecipitates of SLIM-DC (FIG. 5A), which we subsequently identified as the RING finger ubiquitin ligase CbI (FIG. 5B). CbI has been previously shown to be a negative regulator in TCR signaling pathway and to constitutively interact with the C-terminal region of SLAP as demonstrated in both a yeast two hybrid system and mammalian cells. However, the association between SLIM and CbI in B cells was inducible following antigen receptor stimulation, suggesting SLIM and SLAP may utilize different mechanisms to recruit other signaling partners. It would be possible for SLIM to function as an inhibitory adapter by either recruiting a negative regulator into the signaling pathway and/or by directly blocking the function of a positive regulator. Our results suggest that SLIM functions via the former mechanism in antigen receptor signaling cascades.

SLIM associates with tyrosine phosphorylated proteins in B cells. Sorted tTA-BJAB cells infected with epitope-tagged wild type SLIM, SLIM-myr or SLIM-DC were stimulated with anti-Igm F(ab′)₂ for 2 mins, lysed and SLIM was immunoprecipitated using anti-FLAG antibodies. Immunoprecipitated proteins were subjected to SDS-PAGE and immunoblotted with anti-phosphotyrosine antibodies. SLIM associates with tyrosine phosphorylated proteins of approximately 110 and 70 kDa after antigen stimulation. The AC mutant lacks the 110 kDa SLIM-associated phosphoprotein. Lower panel: Reprobe with anti-FLAG. (FIG. 5)

SLIM interacts with CbI in B cells. Wild type SLIM, SLIM-myr or SLIM-DC were immunoprecipitated as in A and immunoblotted with anti-CbI antibodies. WT SLIM and SLIM-myr but not SLIM-DC associate with CbI after antigen stimulation. (FIG. 5)

We claim:

1. A SLIM nucleic acid encoding a SLIM protein, comprising a nucleic acid sequence having at least about 90% identity to the nucleic acid sequence set forth in FIG. 2, wherein said SLIM protein comprises an N-terminal myristylation sequence, an N-terminal SH2 domain, and an N-terminal SH3 domain and will bind to CbI.

2. The SLIM nucleic acid according to claim 1, wherein said SLIM protein lacks a tyrosine kinase domain.

3. The SLIM nucleic acid according to claim 2, further comprising the nucleic acid sequence set forth in FIG. 2.

4. A SLIM nucleic acid encoding a SLIM protein, comprising a nucleic acid sequence having at least about 90% identity to the nucleic acid sequence set forth in FIG. 2, wherein said SLIM protein comprises an N-terminal myristylation sequence and an N-terminal SH2 domain and is unable to bind to CbI.

5. A SLIM nucleic acid encoding a SLIM protein, comprising a nucleic acid sequence encoding an amino acid sequence having at least about 90% identity to the amino acid sequence set forth in FIG. 2.

6. A SLIM protein, comprising an amino acid sequence having at least about 90% identity to the amino acid sequence set forth in FIG. 2, wherien said SLIM protein comprises an N-terminal myristylation sequence, an N-terminal SH2 domain, and an N-terminal SH3 domain and will bind to CbI.

7. The SLIM protein according to claim 6, further comprising the amino acid sequence set forth in FIG. 2.

8. A SLIM protein, comprising an amino acid sequence having at least about 90% identity to the amino acid sequence set forth in FIG. 2, wherein said SLIM protein comprises an N-terminal myristylation sequence and an N-terminal SH2 domain and is unable to bind to CbI.

9. A method for screening for a bioactive agent capable of binding to SLIM, comprising:
a) contacting a SLIM protein and a candidate agent; and
b) determining the binding of candidate bioactive agent to SLIM protein;
wherein said SLIM protein comprises an amino acid sequence having at least about 95% identity to the amino acid sequence set forth in FIG. 2.

10. A method for screening for a bioactive agent capable of modulating SLIM binding, comprising:
a) combining a SLIM protein, a candidate bioactive agent and CbI; and
b) determining the binding of CbI to SLIM in the presence of candidate bioactive agent;
wherein said SLIM protein comprises an amino acid sequence having at least about 95% identity to the amino acid sequence set forth in FIG. 2 and wherein said SLIM protein will bid to CbI in the absence of candidate bioactive agent.

11. A method for screening for a bioactive agent capable of modulating lymphocyte activation, comprising:
a) contacting a candidate bioactive agent to a lymphocyte comprising a recombinant nucleic acid encoding a SLIM protein;
b) inducing activation of said lymphocyte; and
c) determining the activation of said lymphocyte in the presence and absence of said candidate bioactive agent;
wherein said SLIM protein comprises an amino acid sequence having at least about 95% identity to the amino acid sequence set forth in FIG. 2, and wherein a difference in the activation of said lymphocyte in the presence and absence of said candidate bioactive agent indicates that said candidate bioactive agent is capable of modulating lymphocyte activation.

12. The method according to claim 11, wherein said SLIM protein comprises the amino acid sequence set forth in FIG. 2.

13. The method according to claim 11, wherein lymphocyte activation is done by activating antigen receptor in said lymphocyte.

14. The method according to claim 11, wherein determining the activation of said lymphocyte comprises determining the activity of a nuclear factor in activated T cells (NFAT) responsive promoter.

15. The method according to claim 11, wherein determining the activation of said lymphocyte comprises determining the expression of CD69.

16. A method for screening for a bioactive agent capable of modulating the ubiquitination of a CbI target protein, comprising:
a) combining SLIM, CbI, ubiquitin or polyubiquitin, and a CbI target protein; and
b) determining the level of ubiqitination of CbI target protein in the presence and absence of candidate bioactive agent;
wherein said SLIM protein comprises an amino acid sequence having at least about 95% identity to the amino acid sequence set forth in FIG. 2 and will bind to CbI and CbI target protein in the absence of candidate agent, wherein a change in the level of ubiquitination of CbI target protein in the presence of candidate agent indicates that said candidate bioactive agent is capable of modulating the ubiquitination of a CbI target protein.
